(19) United States
(12) Patent Application Publication
(10) Pub. No.: US 2012/0197277 A1
(43) Pub. Date: Aug. 2, 2012

(54) VASCULAR PLAQUE REMOVAL SYSTEMS, DEVICES, AND METHODS

(76) Inventor: Curtiss T. Stinis, San Diego, CA (US)

(21) Appl. No.: 13/363,099

(22) Filed: Jan. 31, 2012

Related U.S. Application Data
(60) Provisional application No. 61/438,547, filed on Feb. 1, 2011.

(51) Int. Cl. A61B 17/221

(52) U.S. Cl. 606/159; 606/190

(57) ABSTRACT

Systems, devices, and methods for removing plaque from a patient's vasculature. In one example, a medical article includes a catheter body and a dissection tip. The dissection tip can move between at least a first position and a second position and can be biased to expand radially from a longitudinal axis of the medical article when the dissection tip is moved from the first position to the second position. In one example, a dissection member includes a dissection tip and a receiving space. The dissection tip can radially adjust to circumferentially maneuver between a core of plaque and a patient's vasculature. The receiving space can receive at least a portion of a core of plaque that passes through the dissection tip.

### CROSS-REFERENCE TO RELATED APPLICATIONS

This application claims the benefit of U.S. Provisional Application No. 61/438,547 filed on Feb. 1, 2011, titled “VASCULAR PLAQUE REMOVAL SYSTEMS, DEVICES, AND METHODS,” which is hereby expressly incorporated by reference in its entirety.

### BACKGROUND

1. Field

Embodiments disclosed herein relate generally to systems, devices, and methods for treating stenosed blood vessels. More specifically, certain embodiments concern systems, devices, and methods that can be implemented to perform an endovascular endarterectomy procedure in a patient to treat stenosis caused, at least in part, by atherosclerosis.

2. Description of the Related Art

Atherosclerosis can be caused by the accumulation of plaque (e.g., atherosclerotic tissue) inside a person's vasculature. Over time, the accumulated plaque can result in a partial or total occlusion of one or more blood vessels resulting in coronary artery disease, peripheral vascular disease, and/or cerebral vascular disease. Atherosclerosis can be treated by various surgical procedures, for example, balloon angioplasty, atherectomy, and/or inserting one or more intravascular stents, to open up the stenosed blood vessel.

### SUMMARY

The systems, devices, and methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description of Certain Embodiments,” one will understand how the features of the devices and methods disclosed herein provide advantages over other known devices and methods.

In one embodiment, a method may include, for example, introducing a medical article into a patient's vasculature. The medical article may include, for example, a radially adjustable dissection tip, a receiving space disposed proximal to the dissection tip, and an aspiration lumen disposed proximal to the receiving space. The method may also include, for example, positioning the dissection tip between a volume of plaque tissue and an outer wall of the patient's vasculature and receiving the volume of plaque tissue in the receiving space.

In certain aspects, the method may also include, for example, advancing the medical article distally through the patient's vasculature. The medical article may be advanced distally over a guidewire or may be advanced distally without the use of a guidewire. The medical article can include, for example, a severing element. The severing element may have a longitudinal length that does not extend distally beyond the most distal edge of the dissection tip. The severing element may be configured to expand radially when the dissection tip is expanded radially and/or may be configured to rotate relative to the dissection tip when the dissection tip is expanded radially. The severing tip may also not expand.

In other aspects, the method may also include, for example, aspirating at least a portion of the received volume of plaque tissue from the patient's vasculature. At least a portion of the received volume of plaque tissue may be aspirated through an aspiration lumen. In some aspects, positioning the dissection tip between the volume of plaque tissue and the outer wall of the patient's vasculature includes positioning the dissection tip such that the dissection tip is disposed circumferentially about the volume of plaque tissue. The radially adjustable dissection tip may be introduced into the patient's vasculature in a non-deployed position. The radially adjustable dissection tip may be deployed prior to positioning the dissection tip between a volume of plaque tissue and an outer wall of the patient's vasculature. The outer wall of the patient's vasculature may include the endothelium of the blood vessel, the intima of the blood vessel, the subintimal space of the blood vessel, and/or the medical of the blood vessel. The radially adjustable dissection tip may be configured to reduce or expand according to the size of the patient's vasculature. The radially adjustable dissection tip may be configured to automatically reduce in radial size as it is introduced into a narrowing blood vessel. In some aspects, the radially adjustable dissection tip may reduce its actual size as the medical article is advanced distally through the patient's vasculature.

In another embodiment, a medical article may include, for example, a catheter body and a dissection tip. The catheter body may have a distal end, a proximal end, and a longitudinal axis extending therebetween. The dissection tip may be configured to move between at least a first position and a second position. The dissection tip may be disposed at least partially within the catheter body in the first position and the dissection tip may be disposed distal to the distal end of the catheter body in the second position. The dissection tip may be biased to expand radially from the longitudinal axis when the dissection tip is moved from the first position to the second position.

In certain aspects, the medical article may include, for example, a severing element configured to expand radially from the longitudinal axis when the dissection tip is moved from the first position to the second position. The severing element may be configured to rotate relative to the catheter body when the dissection tip is in the second position. The severing element may be configured to rotate relative to the dissection tip when the dissection tip is in the second position. At least a portion of the severing element may be disposed within the catheter body when the dissection tip is in the first position. The severing element may be disposed proximal to the most distal point of the dissection tip when the dissection tip is in the second position. At least a portion of the severing element may be disposed distal to the catheter body when the dissection tip is in the second position.

In other aspects, the medical article may include, for example, an ultrasonic transducer configured to transmit ultrasound energy. At least a portion of the ultrasonic transducer may be disposed distal to the catheter body when the dissection tip is in the second position. The ultrasonic transducer may be configured to transmit ultrasound energy away from the longitudinal axis. The dissection tip may be configured to adjust its radial size according to the size of a blood vessel into which it is introduced. The dissection tip may be configured to decrease its radial size as it is moved into a blood vessel that is more narrow in radial size. The medical article may also include, for example, a plurality of struts coupled to the dissection tip. At least a first portion of each strut may be disposed within the catheter body when the dissection tip is in the second position. At least a second portion of each strut may be disposed distal to the catheter body when the dissection tip is in the second position.

In some aspects, the medical article may include, for example, a casing layer disposed circumferentially around at least a portion of the dissection tip. The casing layer may define a receiving space configured to receive a volume of plaque tissue when the dissection tip is in the second position. At least a portion of the severing element may be disposed within the receiving space. At least a portion of the casing layer may form a frusto-conical shape when the dissection tip is in the second position. The casing layer may include a hydrophilic material, for example, latex or Mylar. Each strut may include steel, for example, spring steel or stainless steel. The dissection tip may include a shape memory alloy, for example, a metal alloy including at least nickel and titanium.

In other aspects, the severing element may be biased to expand radially from the longitudinal axis when the dissection tip is moved from the first position to the second position. At least one of the plurality of struts may bias the severing element to expand radially from the longitudinal axis when the dissection tip is moved from the first position to the second position. The dissection tip may extend substantially parallel to the longitudinal axis when the dissection tip is in the second position. The medical article may also include, for example, a shaft extending at least partially through the catheter body and parallel to the longitudinal axis. At least a portion of the severing element may be configured to slide along at least a portion of the shaft. The severing element may include a first collar configured to slide along at least a portion of the shaft. The first collar may be configured to rotate about the shaft. The shaft may include a guidewire lumen extending at least partially therethrough. The shaft may include a drive shaft configured to rotate the severing element relative to the catheter body.

In yet another embodiment a dissection member may have a proximal end, a distal end, and a longitudinal axis extending therebetween. The dissection member may include, for example, a dissection tip and a receiving space. The dissection tip may be configured to radially adjust in order to circumferentially maneuver between a core of plaque and a patient's vasculature. The receiving space may be disposed proximal to the dissection tip and configured to receive at least a portion of a core of plaque that passes through the dissection tip. In certain aspects, the dissection tip may have a longitudinal length configured to disposed the longitudinal axis of the dissection member substantially parallel to the portion of the patient's vasculature that the dissection tip is disposed within. The dissection member may also include, for example, a severing element configured to sever a portion of plaque tissue that passes through the dissection tip. The severing element may be configured to radially adjust along with the dissection tip. The severing element may be configured to rotate relative to the dissection tip. The severing element may not be configured to expand.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1A is a partially cut away perspective view of one example of a blood vessel.

FIG. 1B is a side view of one example of a portion of a patient's vasculature including two stenosed sections.

FIG. 1C is a cross-section of the patient's vasculature of FIG. 1B taken along line 1C-1C.

FIG. 1D is a cross-section of the patient's vasculature of FIG. 1B taken along the line 1D-1D.

FIG. 1E is a cross-section of the patient's vasculature of FIG. 1B taken along the line 1E-1E.

FIG. 2A is a perspective view of a portion of one non-limiting example of an embodiment of a medical article for use in performing an endovascular endarterectomy procedure.

FIG. 2B is a side view of the medical article of FIG. 2A.

FIG. 2C is a cross-section of the medical article of FIG. 2B taken along line 2C-2C.

FIG. 2D is an end view of the medical article of FIG. 2A.

FIG. 2E is a side view of a proximal portion of the medical article of FIG. 2A.

FIGS. 3A-3C are side views schematically illustrating the use of the medical article of FIGS. 2A-2E in performing an endovascular endarterectomy procedure in an example blood vessel.

FIGS. 4A-4C are side views schematically illustrating the use of the medical article of FIGS. 2A-2E in performing an endovascular endarterectomy procedure in an example blood vessel.

FIGS. 5A-5C are side views schematically illustrating the use of the medical article of FIGS. 2A-2E in performing an endovascular endarterectomy procedure in an example blood vessel.

FIGS. 6A-6C are side views schematically illustrating the use of the medical article of FIGS. 2A-2E in performing an endovascular endarterectomy procedure in an example blood vessel.

FIG. 7A is a perspective view of a portion of one non-limiting example of an embodiment of a medical article for use in separating plaque from a patient's vasculature.

FIG. 7B is a perspective view of a portion of one non-limiting example of an embodiment of a medical article for use in separating plaque from a patient's vasculature.

FIG. 8 is a perspective view of a portion of another non-limiting example of an embodiment of a medical article for use in separating plaque from a patient's vasculature.

FIGS. 9A-9C are side views schematically illustrating the use of the medical article of FIGS. 2A-2E in removing a thrombus from an example blood vessel.

### DETAILED DESCRIPTION OF CERTAIN EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Atherosclerosis can result from the accumulation of plaque inside a patient's vasculature. This accumulation of plaque can result in stenosis or a narrowing of one or more lumens within the patient's vasculature which can cause various complications, for example, infarctions throughout the patient's body (e.g., a myocardial infarction) and/or claudication in certain areas of the body. Atherosclerosis can be fatal and is currently the most prominent cause of death in the United States. Atherosclerosis can be treated by various open surgical procedures and various endovascular procedures (e.g., procedures during which a medical article is inserted into a blood vessel).

Some examples of open surgical procedures to treat atherosclerosis include bypass surgery, open endarterectomy surgery, and surgical remote endarterectomy. In bypass surgery, arteries or veins from elsewhere in a patient's body are grafted to diseased portions of the patient's vasculature to bypass stenosed portions of a blood vessel, for example, atherosclerotic narrowings or blockages in an artery. In some examples, synthetic lumens can be implanted into the patent to bypass the stenosed portions of the blood vessel. In open endarterectomy surgery, a diseased blood vessel is opened with an incision and plaque is physically separated from the blood vessel and removed. Thus, open endarterectomy surgeries are limited to blood vessels and blockages that are readily accessible and close to the skin, for example, the carotid artery, such that the blood vessel can be opened and the plaque can be removed through the opening. In remote endarterectomy surgery, a fixed diameter medical article is inserted through an open incision into a blood vessel and the medical article is advanced distally to strip plaque from the blood vessel. However, because the medical article has a fixed diameter it can stretch the blood vessel and cause barotrauma which promotes restenosis (e.g., the reoccurrence of stenosis). In other cases, the medical article may be too small for a given blood vessel such that the medical article is incapable of stripping plaque from the wall of the blood vessel. Therefore, these existing surgical procedures are limited to certain sized blood vessels that are readily accessible to a healthcare professional and these procedures involve open surgery which has increased risk and recovery time.

Some examples of endovascular procedures to treat atherosclerosis include angioplasty, stenting, and atherectomy procedures. These procedures are performed via a small catheter inserted directly into a blood vessel without an open surgical incision. In angioplasty procedures, a balloon catheter may be advanced over a guidewire to a narrowed or blocked portion of a blood vessel. The balloon may then be inflated to radially compress plaque away from the lumen of the blood vessel to increase blood flow therethrough. When inserting a stent, the stent may be disposed over a balloon catheter such that inflation of the balloon expands the stent radially to hold open the blood vessel. Both angioplasty and inserting a stent apply pressure to the blood vessel to radially compress plaque away from the lumen of the blood vessel. Therefore, these procedures can result in barotrauma of the treated blood vessel which may promote restenosis.

Some examples of atherectomy procedures include directional atherectomy, rotational atherectomy, orbital atherectomy, and laser atherectomy. Atherectomy procedures involve the partial removal of plaque, or atherosclerotic tissue, from a blood vessel using various endovascular medical articles that are advanced through the blood vessel over a guidewire. For example, various directional atherectomy procedures include cutting cores of plaque from a blood vessel and aspirating the cores through a flexible shaft. Most have little or no flexibility in terms of their size, so that they cannot be used in vessels that are too small or they leave behind large amounts of plaque when used in a larger vessel. Also, if too large for the treated vessel, the devices can cause trauma and damage to the vessel. In any case, all current atherectomy procedures leave at least some plaque behind in the treated blood vessel. Removing some plaque from a blood vessel while leaving some plaque behind can result in suboptimal results and/or restenosis. Therefore, existing endovascular procedures require the use of a guidewire and/or leave either plaque or a foreign body (e.g., a stent) behind.

Another challenge with these procedures is the requirement for visualization of the region that is being treated, which typically is done using X-rays or ultrasound. The use of X-rays results in potential exposure to harmful X-rays. To avoid exposure heavy and cumbersome protective articles, usually comprising lead, are worn by medical staff and used by the patients. Nonetheless, the patients are exposed to X-rays. The X-rays permit the medical staff to watch the device (e.g., guidewire, stent, balloon and/or atherectomy tool) inside the patient. However, the use of X-rays can have limitations. For example, the X-rays can be obscured or obstructed by objects within the patient, such as implants (e.g., titanium rods, artificial knees, etc.) and other devices. Thus, the presence or extent of disease around such devices can be obscured and the medical staff must treat such areas blindly. Ultrasound also can be used, but normally cannot be used at the same time as the treatment device. For example, usually the ultrasound device is inserted prior to or after a stent, balloon or atherectomy device is used. In many cases, the atherectomy or balloon device must be removed, then the ultrasound is inserted, then the ultrasound is removed, then the atherectomy, balloon or stent is reinserted. Thus, there is a need for devices that can be used without requiring the use of existing visualization techniques.

Embodiments disclosed herein generally relate to endovascular endarterectomy systems, devices, and methods for performing an endovascular endarterectomy procedure. As used herein, “endovascular endarterectomy” refers to an endovascular procedure where all, or substantially all, of a deposit of plaque tissue is separated and removed from at least a portion of a blood vessel. For example, in some embodiments removal of substantially all of a deposit of plaque can mean removal of from about 70-99.99% of the plaque in the treated portion of the vessel, from about 90-99.99%, from about 95-99.99%, or from about 98-99%. In some embodiments, removal of substantially all of a deposit of plaque can mean removal of about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, or more, of the deposit of plaque. Endovascular endarterectomy procedures according to some embodiments can reduce or minimize restenosis of treated portions of blood vessels by not leaving foreign objects and/or plaque behind and can be performed with medical articles that adjust to the size of the blood vessel to limit or prevent barotrauma of the blood vessel.

Some embodiments disclosed herein relate to medical articles and methods of using the articles in performing an endovascular endarterectomy procedure. Some of these embodiments include a dissection member that can include a dissection tip and a receiving space disposed proximal thereto. The dissection tip can be configured to enter, for example, the subintimal plane between a core of plaque and an outer wall of a blood vessel to circumferentially engulf the core of plaque. In some aspects, the dissection member can be configured to expand and contract radially from a longitudinal axis of the medical article to adjust to the size of the blood vessel that the medical article is introduced into. In some embodiments, the dissection member can be configured to automatically adjust to the size of the blood vessel that the medical article is introduced into and the dissection member can adjust with the blood vessel as the blood vessel expands, contracts, turns, or otherwise changes. The automatic adjustment of the dissection member to fit or conform to the diameter of the vessel or vessel wall can permit the article to be used without the use of X-rays or ultrasound, although such visualization techniques can be used in some embodiments.

Various embodiments of medical articles disclosed herein can include a severing element. In some aspects, the severing element can be configured to rotate relative to the dissection tip to sever at least some portions of the core of plaque that is engulfed within the dissection member. In various embodiments, the severing element can be configured to expand and contract radially from a longitudinal axis of the medical article to adjust along with the dissection member and can be configured to rotate relative to the dissection member. In some embodiments, the severing element can be fixed or may not expand to the full size of the vessel. However, in one non-limiting example of such an embodiment, the dissection member can channel, funnel or direct the material to the severing element so that it can be severed. Thus, the severing element can be configured to sever all or substantially all of the plaque that passes through the dissection member. In some embodiments, the use of a radially adjustable severing element can permit the articles to be used in absence of visualization (e.g., when obstructed or unsafe, etc.) such as X-rays and/or ultrasound, although such visualization techniques can be used in some embodiments, if desired. For example, the adjustable device itself can protect the vessel from being damaged or harmed. For example, in some aspects the device can protect because it conforms to the diameter of the vessel, has relatively blunt leading edges, and does not necessarily require a guidewire to be successfully passed across the blockage to be treated.

In some embodiments, a medical article can include a catheter body having one or more aspiration lumens to allow for the aspiration of the core of plaque and/or the severed material from the patient's blood vessel.

In some embodiments, a medical article can include and/or be associated with an intravenous ultrasound system to image portions of the blood vessel that the medical article is inserted into. For example, in some aspects, the dissection member or other portion of the article can include an ultrasound device. This can permit visualization, for example, prior to removal of blocking material, while material is being removed, and/or after material is removed. The medical article does not have to be removed and/or reinserted in connection with visualization during the treatment procedure.

Certain embodiments disclosed herein relate to methods of performing an endovascular endarterectomy procedure. In some embodiments, a method can include positioning a dissection tip between a volume of plaque and an outer wall of a patient's vasculature such that the volume of plaque is separated from the outer wall and received within the dissection a member. The dissection member can be advanced within the blood vessel and the received volume of plaque can then be at least partially severed from the patient's vasculature and aspirated from the blood vessel. In various embodiments disclosed herein, an endovascular endarterectomy procedure can be performed without the use of a guidewire, which can, for example, prevent blood vessel perforation when extracorporeal imaging is unavailable or not desired. In some embodiments a guidewire can be used, while in others it can be specifically excluded.

Several non-limiting examples of embodiments will now be described with reference to the accompanying figures, wherein like numerals refer to like elements throughout. The terminology used in the description presented herein is not intended to be interpreted in any limited or restrictive manner, simply because it is being utilized in conjunction with a detailed description of certain specific embodiments. Furthermore, embodiments can include several novel features, no single one of which is solely responsible for its desirable attributes or which is essential to practicing the technology herein described.

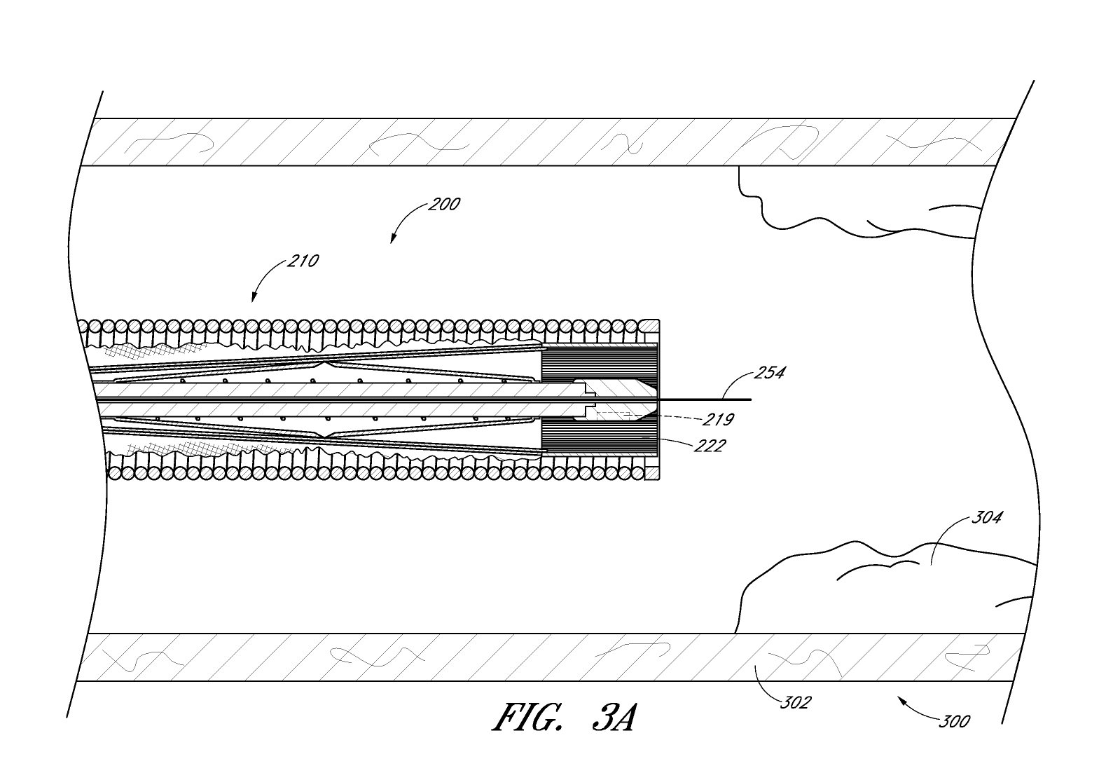

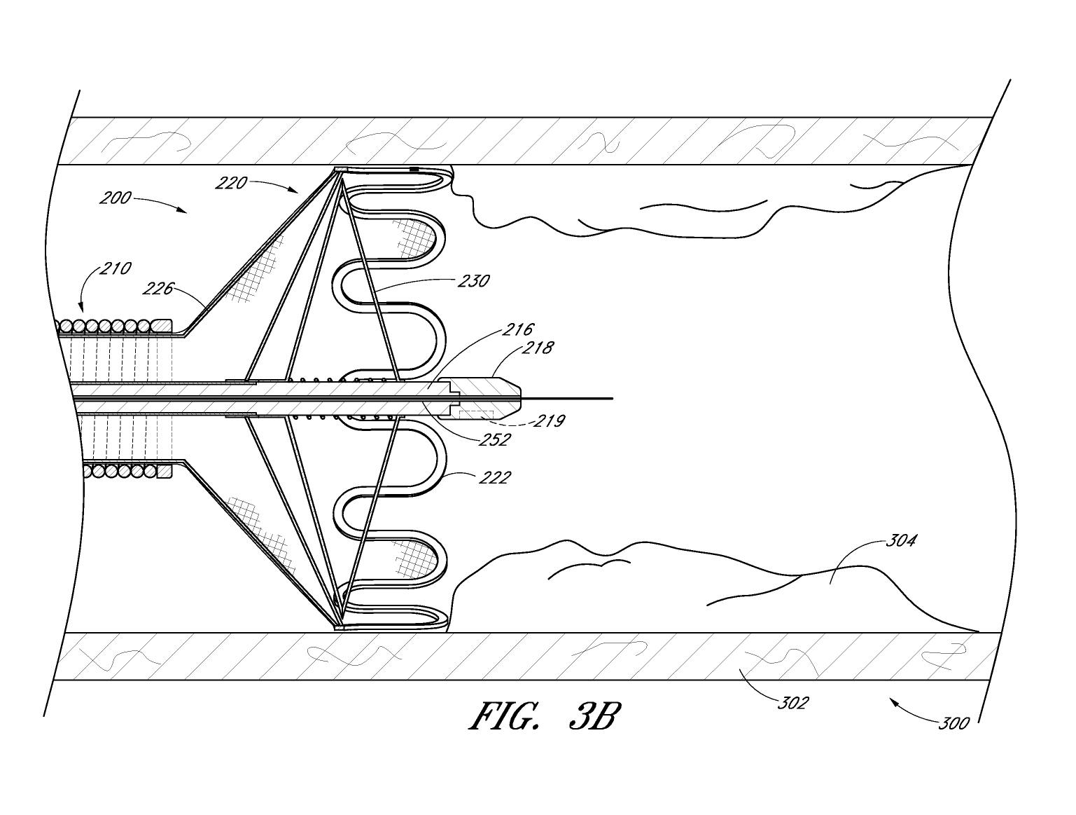

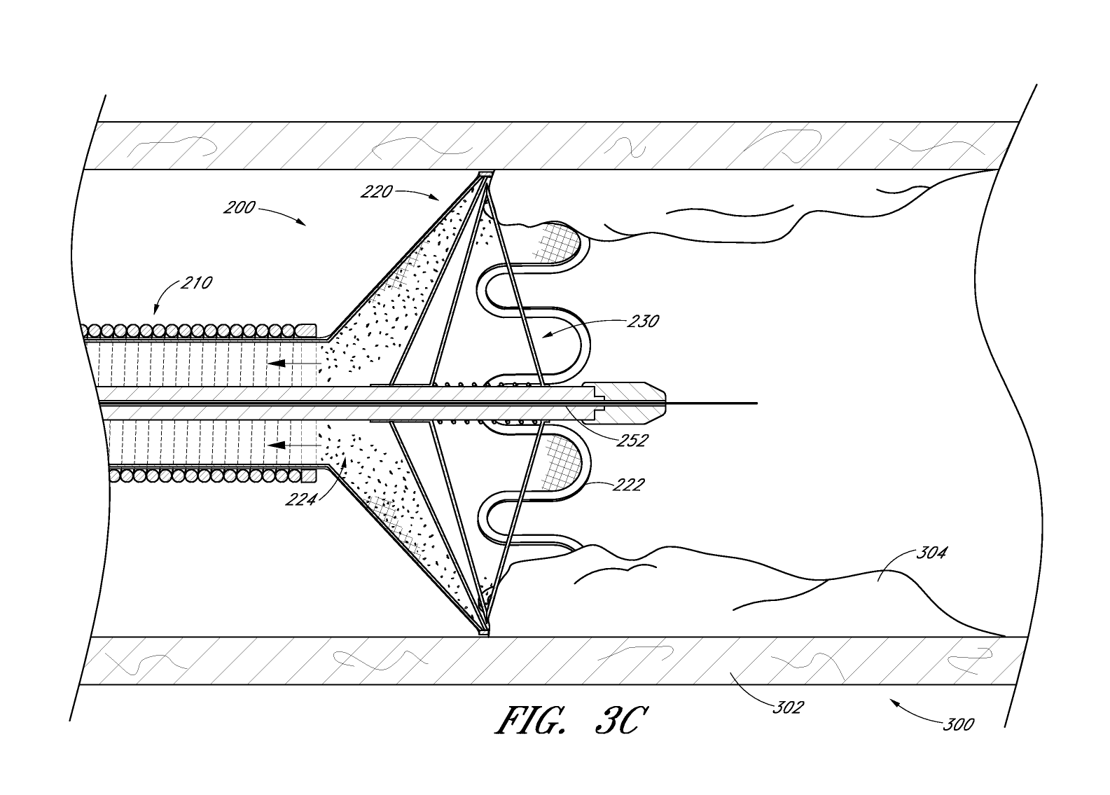

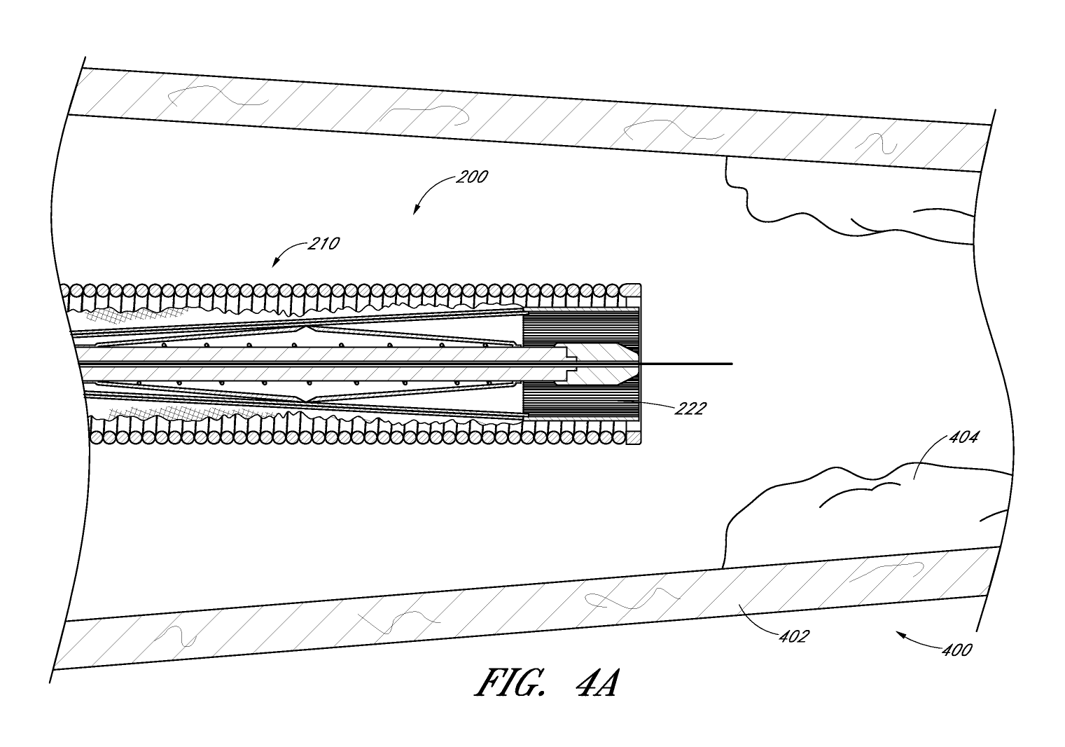

To assist in the description of these components of the systems, devices, and methods described herein (see FIG. 2A), the following coordinate terms are used. A “longitudinal axis” is substantially parallel to the portion of the medical article, as well as parallel to the lumen or channel of the blood vessel. A “lateral axis” is normal to the longitudinal axis, as seen in FIG. 2A. A “transverse axis” extends normal to both the longitudinal and lateral axes. In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. As used herein, “substantially parallel” can refer to two or more lines or directions that do not intersect or that define an angle of about 15° or less at an intersection. For example, in some embodiments, substantially parallel lines or directions can mean lines or directions that do not intersect or that define an angle of about 15°, 14°, 13°, 12°, 11°, 10°, 9°, 8°, 7°, 6°, 5°, 4°, 3°, 2°, 1°, or fewer, at an intersection of the lines or directions.

“Connected” and “coupled,” and variations thereof, as used herein include direct connections, such as being glued or otherwise fastened directly to, on, within, etc. another element, as well as indirect connections where one or more elements are disposed between the connected elements.

“Secured” and variations thereof as used herein include methods by which an element is directly secured to another element, such as being glued or otherwise fastened directly to, on, within, etc. another element, as well as indirect means of securing two elements together where one or more elements are disposed between the secured elements.

Movements which are “counter” are movements in the opposite direction. For example, if the medical article is rotated clockwise, rotation in a counterclockwise direction is a movement which is counter to the clockwise rotation. Similarly, if the medical article is moved substantially parallel to the longitudinal axis of the blood vessel in a distal direction, movement substantially parallel to the longitudinal axis in a proximal direction is a counter movement.

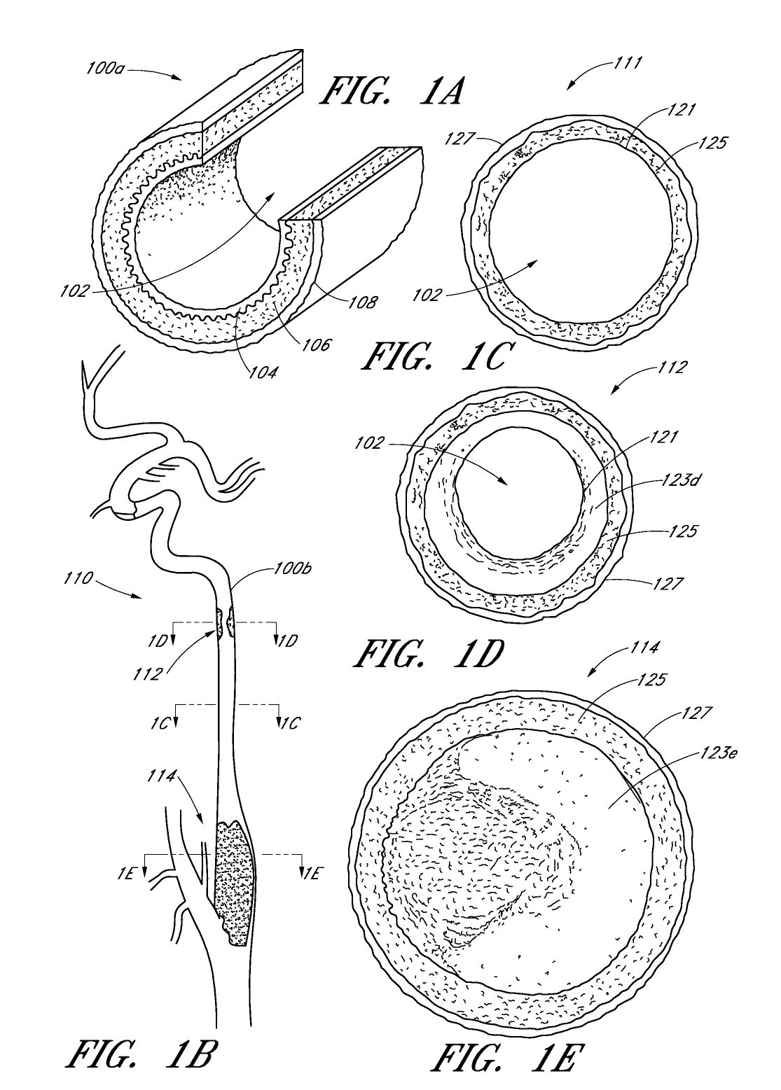

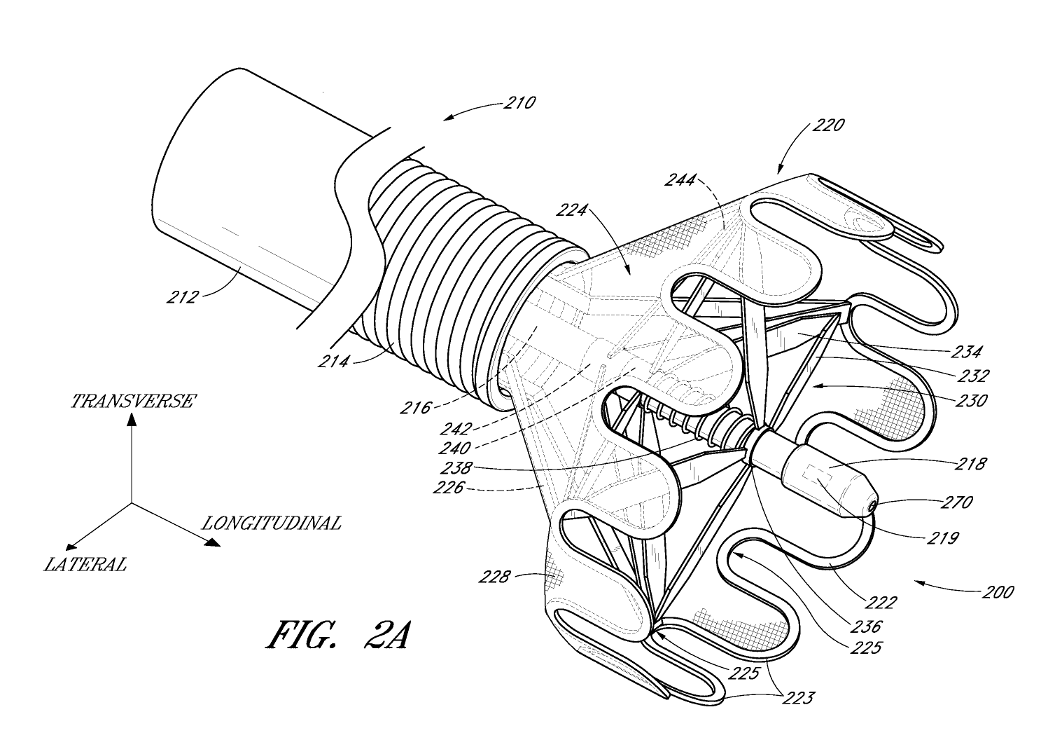

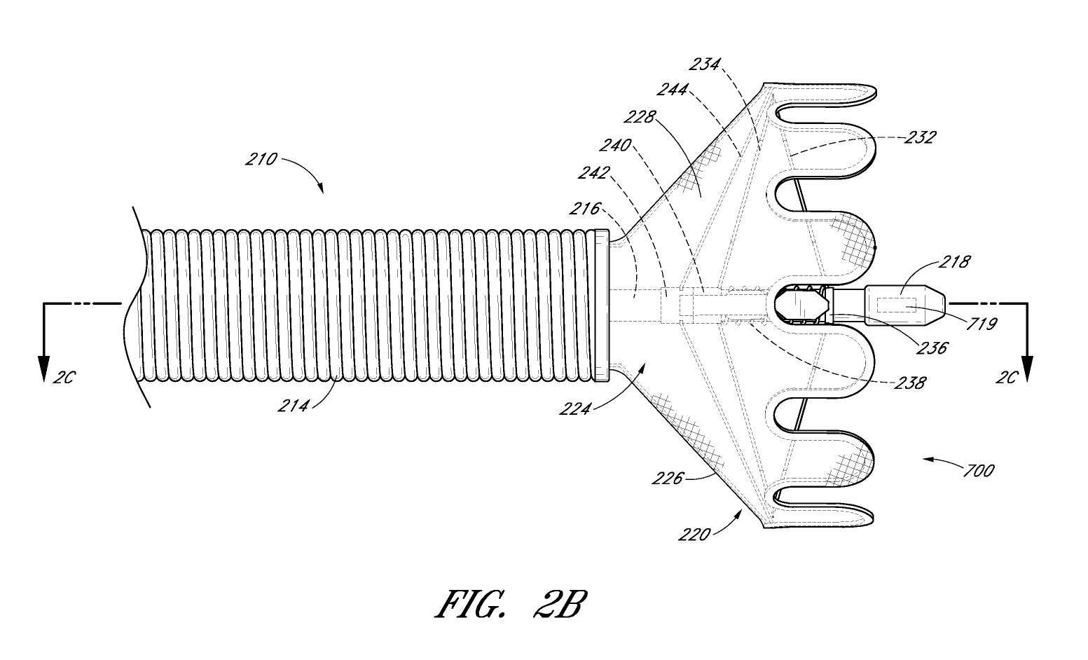

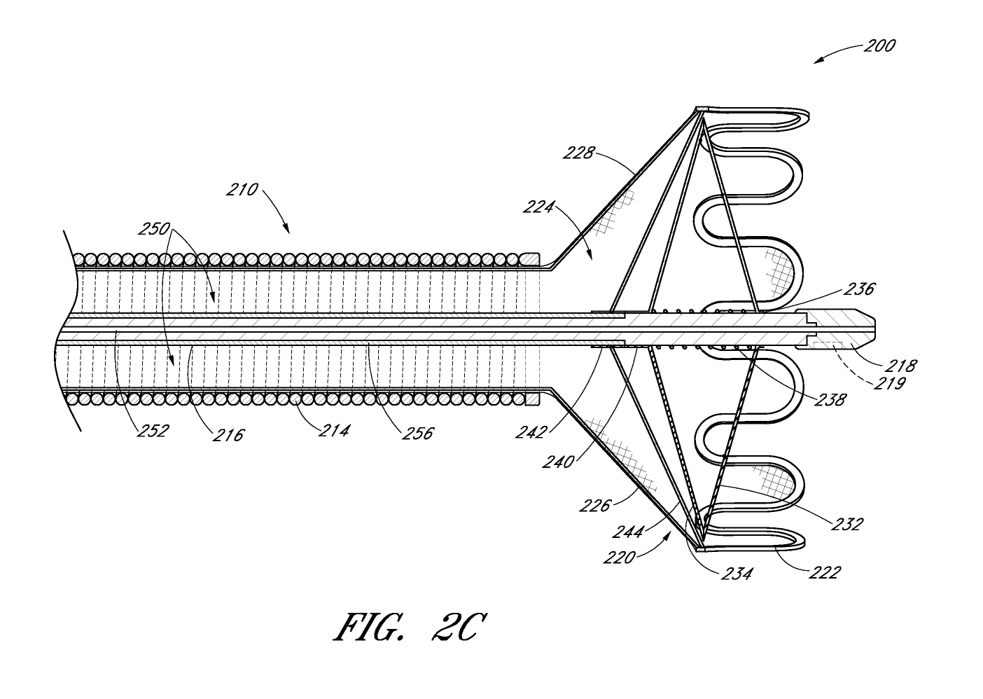

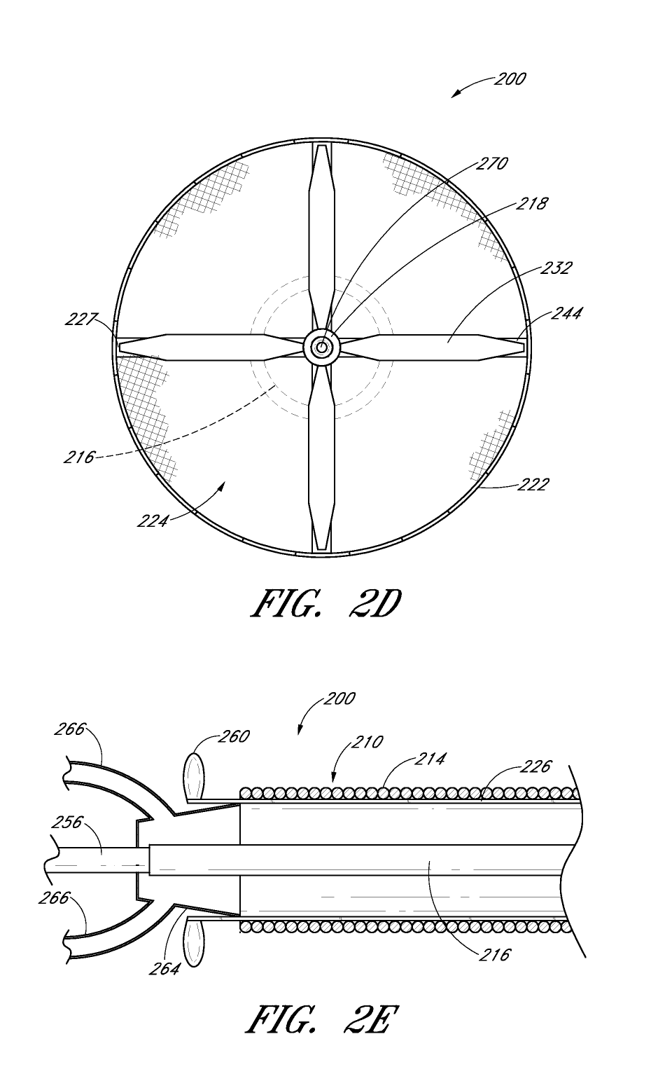

FIG. 1A is a partially cut away perspective view of one example of a blood vessel. The blood vessel 100a includes a central lumen 102 through which blood may pass and an outer layer of connective tissue or adventitia 108 that surrounds the blood vessel 100a. The blood vessel 100a also includes an inner layer or intima 104 and a middle layer or media 106 disposed between the adventitia 108 and the intima 104. As discussed in more detail below with reference to FIGS. 1B-1E, fatty materials, for example, cholesterol, can build up within the blood vessel 100a causing atherosclerosis and stenosis of the blood vessel 100a.

Turning now to FIG. 1B, a side view of an illustration of a portion of a patient's vasculature including two stenosed sections is schematically illustrated. The portion of the patient's vasculature 110 includes an artery 100b having a first stenosed section 112 and a second stenosed section 114.

FIG. 1C is a cross-section of a non-stenosed section 111 of FIG. 1B taken along the line 1C-1C. As with the example blood vessel of FIG. 1A, the non-stenosed section 111 includes a central lumen 102 through which blood may pass and an outer layer of connective tissue or adventitia 127 that surrounds the section 111 of the blood vessel 110. The blood vessel 110 also includes an inner layer or intima 121 and a middle layer or media 125 disposed between the adventitia 127 and the intima 121.

FIG. 1D is a cross-section of the first stenosed section 112 of FIG. 1B taken along the line 1D-1D. The stenosed section 112 depicts a reduced lumen 102 through which blood flows. The stenosed section 112 also includes a region of subintimal thickening 123d, which may include a layer of plaque, and which is disposed between the intima 121 and media 125 of the blood vessel 100b. The region of subintimal thickening 123d in the blood vessel 100b narrows the channel or lumen through which blood may pass through (e.g., causes stenosis). Additionally, the region 123d can rupture and cause a thrombus to form that may travel through the patient's vasculature. A dislodged thrombus may become lodged in a narrow portion of vasculature resulting in necrosis of tissue. The stenosed section 112 schematically illustrated in FIGS. 1B and 1D can be treated using various existing endovascular procedures. However, these procedures can result in barotrauma of the blood vessel 100b (e.g., caused by scraping of the vessel during surgical remote endarterectomy or by a balloon with angioplasty) and/or can leave behind a foreign object (e.g., a stent) and/or a portion of plaque in the region of thickening 123d, which would promote restenosis. Alternatively, the region 123d may be completely removed from the stenosed section 112 using the new endovascular endarterectomy systems, devices, and methods disclosed herein.

FIG. 1E is a cross-section of the section stenosed section 114 of FIG. 1B taken along the line 1E-1E. The stenosed section 114 includes a volume of plaque 123e that completely occludes blood flow through the blood vessel 100b at the second stenosed section 114. This complete occlusion can result in an infarction and/or necrosis of another tissue. The stenosed section 114 schematically illustrated in FIGS. 1B and 1E can be treated using subintimal angioplasty procedures where a balloon catheter is advanced between the plaque 123e and the adventitia 127 of the blood vessel 114. However, subintimal angioplasty procedures require the use of a guidewire to position the balloon catheter and stent between the plaque 123e and the adventitia.

In some circumstances, the compression of plaque within the confined space of a blood vessel can result in various deleterious effects (e.g., barotrauma). Further, completely occluded arteries cannot always be visualized and since their path may not be obvious, blindly advancing a guidewire may perforate the blood vessel, leading to complications. Additionally, open surgical procedures can be utilized to treat a complete occlusion, for example, stenosed section 114 of FIG. 1E. However, as discussed above, these procedures require that the occluded section of the blood vessel be readily accessible to a healthcare professional (e.g., proximal to the skin of a patient) and these procedures can be more invasive and risky than endovascular procedures. Alternatively, the plaque 123e may be completely removed from the stenosed section 114 using the new endovascular endarterectomy systems, devices, and methods disclosed herein.

Thus, embodiments disclosed herein include medical articles and methods that can be used to endovascularly treat the stenosed sections 112, 114 of FIGS. 1B, 1D, and 1E without leaving foreign objects and/or plaque behind, without causing significant barotrauma to the blood vessel 100b, and without requiring the use of a guidewire. Furthermore, some embodiments permit the procedures to be performed without the use of X-ray or ultrasound visualization, if desired. Thus, embodiments disclosed herein may effectively treat diseased arteries and decrease the likelihood of restenosis as compared to existing methods of treating atherosclerosis.

FIG. 2A is a perspective view of a portion of one example of an embodiment of a medical article for use in performing an endovascular endarterectomy procedure. FIG. 2B is a side view of the medical article of FIG. 2A. The medical article 200 of FIGS. 2A and 2B includes a catheter body 210 and a dissection member 220 configured to move radially and longitudinally relative to the catheter body 210 and schematically illustrated in FIGS. 2A and 2B as distal to the catheter body 210. The catheter body 210 can comprise an elongated tubular shape defining one or more internal lumens. In some embodiments, the catheter body 210 can be flexible enough to be steered through a tortuous portion of a patient's vasculature yet may be rigid enough to be pushed distally through a given lumen. Thus, in some embodiments, the catheter body 210 can include a flexible coil body 214. The catheter body can optionally by coated, for example, by a hydrophilic coating 212 to assist in catheter passage across stenoses.

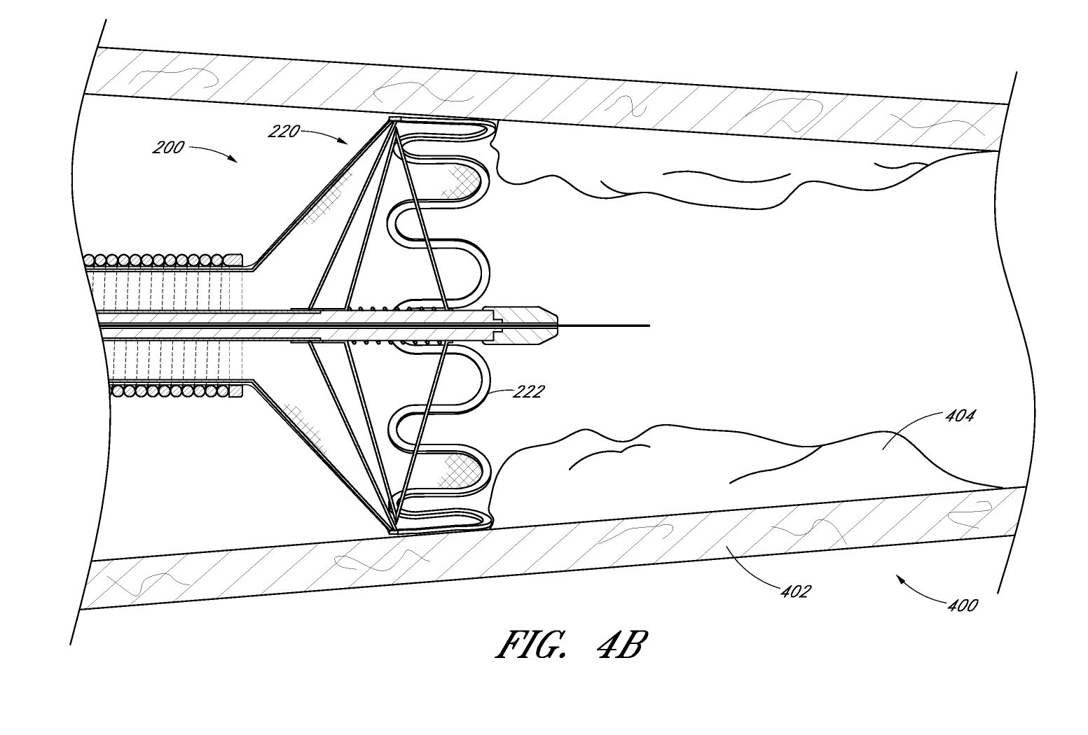

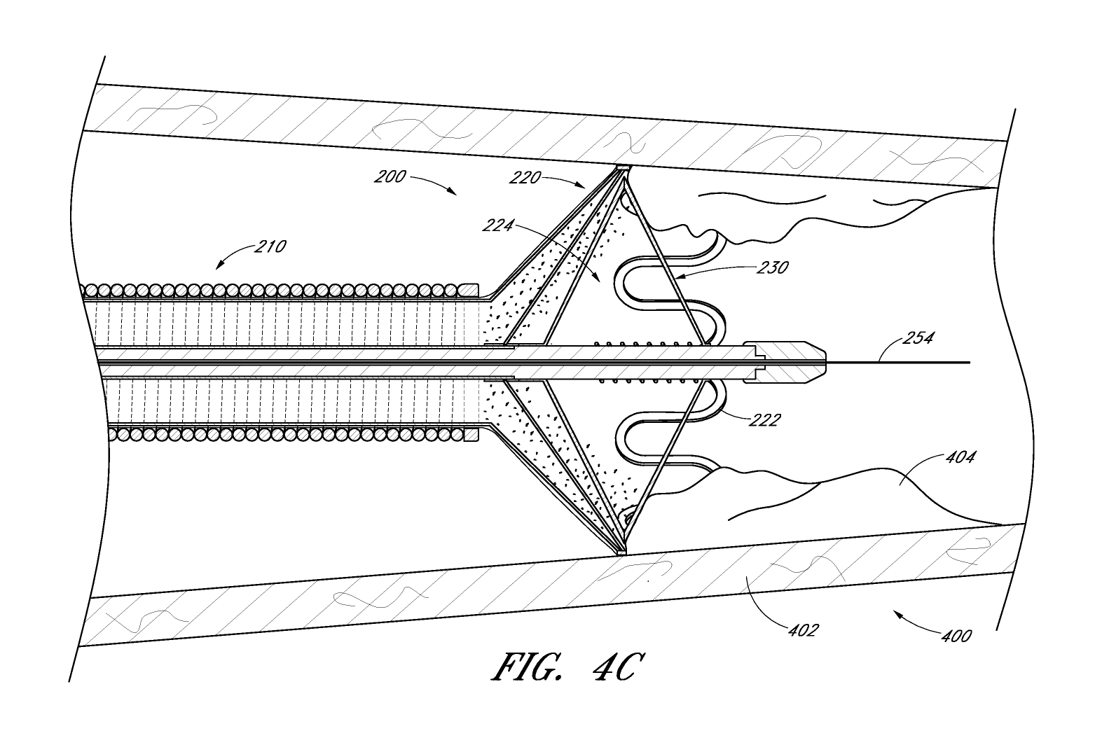

As discussed in more detail below with reference to FIGS. 3A-6C, the dissection member 220 can be configured to adjust radially (e.g., to expand or contract radially) from the longitudinal axis of the medical article 200 between at least a first position and a second position (shown in FIGS. 2A-2C). In some embodiments, the dissection member 220 can be at least partially disposed within the catheter body 210 when it is in the first position and can be disposed distal to a distal end of the catheter body 210 when it is in the second position. The dissection member 220 can include a dissection tip 222 and a receiving space 224 disposed proximal thereto. The dissection tip 222 can define an opening that provides ingress and egress to the receiving space 224. In some embodiments, the opening can be circular, oval, or irregularly shaped and can be defined by, and/or conform to, the shape of a blood vessel that the dissection tip 222 is disposed within.

In some embodiments, the dissection tip 222 can optionally have one or more fingers 223 that extend substantially parallel to one another and substantially parallel to the longitudinal axis of the medical article 200. In use, the fingers can extend substantially parallel to the walls of a blood vessel such that the medical article 200 can be moved longitudinally relative to the blood vessel. In some embodiments, the fingers 223 can be curvilinearly shaped and can be connected to one another by one or more arc segments 225. However, in other embodiments, the fingers 223 can be differently shaped, for example, they may be polygonal and connected to one another by curved segments and/or linear segments. In some embodiments, the dissection tip 222 may not comprise fingers and may be differently shaped, for example, the dissection tip 222 can include teeth, wedges, or other objects that are shaped differently than the fingers 223. Additionally, in other embodiments, the distal end of the dissection tip 222 can be planar. In some embodiments, the dissection tip 222 can comprise a flexible alloy, for example, a nickel-titanium alloy, such that the dissection tip 222 is somewhat rigid yet can expand and flex radially between the first position and the second position.

Still referring to FIGS. 2A and 2B, in some embodiments, the medical article 200 can include one or more struts 226. The struts 226 can be configured to move longitudinally relative to the catheter body 210 and can be disposed at least partially within the catheter body 210 as shown. For example, in one embodiment, the struts 226 can slide within the catheter body 210. In some embodiments, the stru is 226 can comprise a flexible metal, for example, steel, stainless steel, or spring steel, having a pre-formed memory such that the distal ends of the struts 226 are configured to deflect radially away from the longitudinal axis of the medical article 200 when the struts 226 are extended distally from the distal end of the catheter body 210 (e.g., when the distal ends of the struts 226 are not bounded by the catheter body 210).

The struts 226 can be optionally coupled to a proximal end of the dissection member 220. In some embodiments, a hinge, for example a living hinge, can be disposed between the struts 226 and the dissection tip 222 to rotatably or hingedly couple the struts 226 to the dissection member 220. In this way, distal ends of the struts 226 may be completely disposed within the catheter body 210 when the dissection member 220 is in the first position and the distal ends of the struts 226 can be translated longitudinally through the distal end of the catheter body 210 such that the distal ends of the struts 226 deflect radially away from the longitudinal axis of the medical article 200 upon passing through the distal end of the catheter body 210. The longitudinal movement and radial deflection of the struts 226 relative to the longitudinal axis of the medical article 200 can move the dissection tip 222 between at least the first position and the second position (illustrated in FIG. 2A). Thus, when the struts 226 are moved distally relative to the catheter body 210 within a blood vessel, the struts 226 and the dissection tip 222 may expand radially to contact the inner wall of the blood vessel. In some embodiments, the struts can have a pre-formed memory such that the dissection tip 222 automatically expands to the boundaries of the inner lumen of a blood vessel (e.g., to the surface of the inner wall of the blood vessel) and the size of the dissection tip 222 can vary with the blood vessel as the medical article 200 is advanced and/or retracted therethrough.

With continued reference to FIGS. 2A and 2B, an optional casing layer 228 can be disposed circumferentially around the struts 226 and at least a portion of the dissection member 220. The casing layer 228 can comprise various flexible materials, for example, materials such as latex and/or Mylar, and can comprise various non-flexible materials, for example, rigid composites. In some embodiments, the casing layer 228 can comprise a stretchable plastic or rubber material that is disposed circumferentially around the struts 226 and the dissection member 220. In some embodiments, the casing layer 228 can extend along the longitudinal length of the struts 226 and in other embodiments, the casing layer 228 can extend along a portion of the longitudinal length of the struts 226 that is less than an entire longitudinal length of the struts 226. When the dissection member 220 is in the second position (shown in FIGS. 2A and 2B), the casing layer 228 can at least partially define the receiving space 224 between the distal end of the dissection tip 222 and the distal end of the catheter body 210. The receiving space 224 can be defined by various shapes including, for example, frusto-conical shapes, conical shapes, and/or frustums. As discussed in more detail below, the receiving space 224 can be configured to receive a portion of plaque that has been separated from a blood vessel by the dissection tip 222.

The medical article 200 also can include a severing element 230. Any suitable element can be utilized that at least partially chops, cuts, severs, reduces, grinds, separates, divides, or otherwise breaks up the material that is dislodged by the member 220. Any severing element can be utilized, including those that are commercially available, otherwise publicly known, or those described herein.

Without being limited to the exact configuration, FIG. 2A depicts an example of a severing element that is configured to rotate about a shaft 216 that extends through at least a portion of the catheter body 210. The severing element 230 can include a first set of blades 232 and a second set of blades 234. Each of the first set of blades 232 can optionally be rotatably coupled to a fixed collar 236 that is disposed about the shaft 216 and can also optionally by rotatably coupled to one of the second set of blades 234. Each of the second set of blades 234 can be rotatably coupled to a first slidable collar 240 that is disposed about the shaft 216. The fixed collar 236 can be fixed longitudinally relative to the shaft 216 but configured to rotate about the shaft 216 and the first slidable collar 240 can move longitudinally relative to the shaft 216 and also be configured to rotate about the shaft 216.

In some embodiments, the first and second sets of blades 232, 234 can form a scissor like structure with a variable diameter that can rotate relative to the shaft 216. The first and second sets of blades 232, 234 can be configured to break up material that is dislodged by the member 220 (e.g., tissue, plaque, calcified material, etc.), for example by severing, grinding, cutting, chopping, etc. the material that comes into contact with the blades when the blades rotate relative to the shaft 216. In some embodiments, an extension spring 238 can be disposed about the shaft 216 and can couple the fixed collar 236 to the first slidable collar 240. The optional extension spring 238 can act to bias the first slidable collar 240 toward the fixed collar 236 such that the first and second sets of blades 232, 234 are biased towards one another and towards the dissection member 220 when the dissection member 220 is in the second position. As discussed in more detail below with reference to FIGS. 3A-6C, the severing element 230 can be configured to adjust, for example, to expand radially when the dissection member 220 and dissection tip 222 expand radially and can be configured to rotate about the shaft 216 relative to the dissection member 220 to sever plaque that is received within the receiving space 224.

Also schematically depicted in FIGS. 2A and 2B are connection members 244 that couple each of the struts 226 to a second slidable collar 242. Each of the connection members 244 can comprise a rigid material, for example, steel, stainless steel, or spring steel, can be rotatably coupled at one end to a strut 226, and can be rotatably coupled at an opposite end to the second slidable collar 242. Thus, the connection members 244 can serve to indirectly couple the second slidable collar 242 with the struts 226. In this way, radial expansion or outward deflection of the struts 226 relative to the longitudinal axis of the medical article 200 can slide the second slidable collar 242 distally along the shaft 216. Also, radial contraction or inward deflection of the struts 226 relative to the longitudinal axis of the medical article 200 can slide the second slidable collar 242 proximally along the shaft 216. The second slidable collar 242 can be disposed adjacent to and proximal the first slidable collar 240 and can be configured to abut and/or otherwise engage the first slidable collar 240. Therefore, proximal movement of the second slidable collar 242 can apply a force on the first slidable collar 242 which can result in an extension of the extension spring 238. Similarly, distal movement of the second slidable collar 242 can apply a force on the first slidable collar 242 which can result in a compression of the extension spring 238. Accordingly, the connection members 244 and extension spring 238 can act in concert to adjust the diameter of the severing element 230 relative to the position of the dissection tip 222 and struts 226.

In some embodiments, the struts 226 can be rotatably fixed relative to the connection members 244 and the second slidable collar 242 can also be rotatably fixed relative to the struts 226 and shaft 216. Thus, the severing element 230 can be configured to rotate about the shaft 216 relative to the struts 226, dissection member 220, and second slidable collar 242.

The medical article 200 also optionally can include a tip 218. The tip 218 may include, for example, one or more ultrasonic transducers 219. The tip 218 may include, for example, a distal guidewire aperture 270 that provides access to a guidewire lumen 252 (depicted in FIG. 2C). As depicted, the tip 218 can include both the one or more transducers 219 and the guidewire aperture 270, but in some embodiments may include neither of those elements, one of those elements, both elements and/or additional elements. The optional distal guidewire aperture 270 can provide ingress and egress to an optional guidewire lumen in the shaft 216 (see FIG. 2C) to allow an optional guidewire to slide in and out of the shaft 216. The tip 218 can be disposed at a distal end of the shaft 216 and can extend distal to the distal most edge of the dissection tip 222 or can be disposed proximal to the distal most edge of the dissection tip 222.

In some embodiments, the one or more ultrasonic transducers 219 can be part of an intravascular ultrasound system configured to image portions of a blood vessel that the medical article 200 may be inserted into. In such embodiments, the intravascular ultrasound system can be side looking (e.g., radial to the longitudinal axis of the medical article 200) and/or forward looking (e.g., parallel to the longitudinal axis of the medical article 200). In some embodiments, the shaft 216 can include one or more conductive elements, for example, one or more wires, such that signals may be sent and received by the one or more transducers 219 to control circuitry located proximal to the tip 218 (e.g., proximal to the medical article 200). Thus, the one or more ultrasonic transducers 219 and an associated intravascular ultrasound system can enable a health care professional to position the medical article 200 and dissection member 220 relative to a patient's blood vessel. For example, the one or more ultrasonic transducers 219 can be utilized to position the dissection tip 222 circumferentially around a core of plaque and between the plaque and a wall of a blood vessel.

FIG. 2C is a cross-section of the medical article of FIG. 2B taken along line 2C-2C. As discussed above and schematically illustrated in FIG. 2C, the shaft 216 can optionally include a guidewire lumen 252 extending therethrough. In some embodiments the medical article 200 can be advanced and/or retracted through a patient's vasculature over a guidewire that extends through the medical article 200. However, in other embodiments, the medical article 200 can be advanced and/or retracted through a patient's vasculature without a guidewire. Thus, in some embodiments the medical article 200 can include a guidewire lumen 252 extending longitudinally though the shaft 216 and in other embodiments the medical article 200 does not include a guidewire lumen 252.

Also schematically illustrated in FIG. 2C is a drive shaft 256 disposed at least partially within the shaft 216. The drive shaft 256 can be coupled to the first slidable collar 240 and can be configured to rotatably drive the first slidable collar 240 and severing element 230 about the shaft 216. In some embodiments, the drive shaft 256 can be driven by one or more external motors (not shown) that are operationally coupled to the drive shaft 256. In some embodiments, the operation of the drive shaft 256 can be controlled by a control system (not shown) such that the operation of the drive shaft 256 can be controlled independently of the other features of the medical article 200. In some embodiments, the drive shaft 256 can include one or more lumens extending therethrough and the one or more lumens may receive the guidewire lumen 252. In embodiments without a guidewire lumen 252, the drive shaft 256 can be solid.

Still referring to FIG. 2C, the catheter body 210 can define a lumen 250 extending between a distal end and proximal end of the catheter body 210. In some embodiments, the catheter body 210 can include more than one lumen, for example, two, three, four, five, six, seven, eight, nine, ten, or more lumens. The lumen 250 can at least partially receive various components of the medical article 200, for example, the struts 226, the shaft 216, the drive shaft, 256, the guidewire lumen 252, the severing element 230, the first and second slidable collars 240, 242, the connection members 244, the dissection member 220, and/or the tip 218. The lumen 250 can also be configured to provide for the aspiration of removed material such as atherosclerotic tissue or plaque therethrough. For example, plaque engulfed within the dissection member 220 can be removed, for example, via aspiration by passing into the receiving space 224 defined by the casing layer 228 and then passing into a lumen 250 by which is can be removed by aspiration from the medical article. In some aspects, the material may be severed by the severing element 230 and removed as described above, for example, via the receiving space 224. From the receiving space 224, the severed pieces of plaque may be aspirated from the blood vessel through the lumen 250 and disposed of outside of the patient's body. Thus, plaque that is separated from a blood vessel by the dissection tip 222 can be removed from the patient through the lumen 250. In some embodiments, the lumen 250 may allow for the passage of blood through the catheter body 210.

FIG. 2D is an end view of the medical article of FIG. 2A. As shown in FIG. 2D, the first set of blades 232 can include four blades and the first set of blades 232 can be aligned over the second set of blades 234 (shown in FIGS. 2A-2C) and/or the connection members 244. In other embodiments, the first set of blades 232 and/or the second set of blades 234 can include fewer or more than four blades, for example, 1, 2, 3, 5, 6, 7, 8, 9, or 10. The first set of blades 232 can be angularly spaced from one another in a regular fashion as schematically illustrated in FIG. 2D or can be angularly spaced from one another in an irregular fashion. For example, each of the blades can be spaced from adjacent blades by equal angles or at least one blade can be spaced from adjacent blades by unequal angles. Additionally, in some embodiments, each of the first set of blades 232 may be offset from each of the second set of blades 234 and/or may be offset from each of the connection members 244. Thus, in other embodiments, the second set of blades 234 may be observable from an end view of the medical article 200.

Still referring to FIG. 2D, each of the first set of blades 232 can be spaced apart from the inner surface of the dissection member 220 by a clearance space 277. Additionally, each of the connection members 244 can be directly coupled to the dissection member, thus, providing no clearance therebetween or only enough to permit the movement of the blades. Therefore, the first set of blades 232 can rotate about the shaft 216 in a clockwise or counterclockwise direction with abutting, engaging, or touching the dissection member 220, connection members 244, and/or casing layer 228. In this way, the first set of blades 232 can rotate relative to these components and act to sever or otherwise break up plaque that is received within the receiving space 224.

FIGS. 2A-2D depict one example of a medical article 200 with one example of a severing element, specifically a severing element that includes blades that adjust according to the size of the vessel the device is within or according to the size of the dissection member 220. It should again be noted that other severing element configurations can be utilized. In fact when it is desired to have such an element, any suitable element or means for cutting, chopping, reducing, dividing, grinding, breaking up or otherwise reducing the size of the dislodged material (e.g., plaque, calcified material, tissue, etc.) so that it can be removed from the patient, can be utilized. In some aspects the severing element can have a fixed size and/or shape. In some aspects where the shape and/or size are fixed the article can funnel, channel or otherwise direct the dislodge material toward the severing element so that the material can be reduced. For example, the diameter of the member can decrease, for example, as shown in FIGS. 2A-2D so that the dislodged material will be directed to contact a fixed cutting element, which is not depicted in those figures.

FIG. 2E is a side view of a proximal portion of the medical article of FIG. 2A. The proximal portion of the medical article 200 includes a back piece 264 that is fluidly coupled to aspiration ports 266. In some embodiments, the back piece 264 may act to fluidly plug the proximal end of the catheter body 210. Fluids and/or solid matter that travel through the catheter body 210 can collect in the back piece 264 before being aspirated through the aspiration ports 266. In some embodiments, the catheter body 210 may have a sufficient length such that the entire longitudinal length of the catheter body 210 is not introduced into a patient's vasculature during an endovascular endarterectomy procedure. In such embodiments, the aspiration ports 266 may be fluidly coupled to one or more sources of suction or negative pressure, for example a pump.

FIG. 2E also schematically illustrates how a drive shaft 256 can extend through the shaft 216 within the catheter body 210. One or more seals may be disposed between the drive shaft 256 and the back piece 264 to ensure that aspirated fluid and/or solid matter does not leak out of the back piece 264 at the point of entry for the drive shaft 256. In some embodiments, the proximal ends of the struts 226 can be coupled with a handle or grip 260. In some embodiments, the struts 226 may be configured to rotate relative to the catheter body 210 and to translate longitudinally relative to the catheter body 210. Thus, the handle 260 can facilitate the manipulation of the struts 226 and associated dissection member (shown in FIGS. 2A-2D) relative to the catheter body 210. In some endovascular endarterectomy procedures, the manipulation of the dissection member via the handle 260 can enable a healthcare professional to position the dissection member circumferentially around a deposit of plaque between the deposit of plaque and a blood vessel wall to facilitate the separation and removal of the plaque from the blood vessel.

FIGS. 3A-3C depict side views schematically illustrating the use of the medical article of FIGS. 2A-2E in performing an endovascular endarterectomy procedure in an example blood vessel. FIG. 3A schematically illustrates the medical article 200 inside a blood vessel 300. In some embodiments, the medical article 200 can be advanced and/or retracted through the blood vessel 300 over a guidewire 254. However, as discussed above, in some embodiments the medical article 200 can be advanced and/or retracted through the blood vessel 300 without the use of a guidewire. In some embodiments, with or without the use of a guidewire, the medical article 200 can be introduced into the blood vessel 300 through an incision in an outer wall the patient's vasculature.

The blood vessel 300 can include an outer wall 302 and a layer of plaque, or plaque core, 304 disposed radially inward of the outer wall 302. As discussed above with reference to FIGS. 1A-1D, a blood vessel may include an adventitia layer, a media layer, and an intima layer. However, for clarity, the example blood vessels of FIGS. 3A-6C are schematically depicted with a simplified outer wall 302 and a core of plaque 304 disposed inward therefrom.

Still referring to FIG. 3A, the medical article 200 is schematically illustrated with the dissection tip 222 in a first position. In the first position, the dissection tip 222 is disposed within the catheter body 210. Additionally, the distal ends of the struts 226, which are coupled to the dissection tip 222, are also disposed within the catheter body 210.

Turning now to FIG. 3B, the dissection tip 222 and dissection member 220 are schematically illustrated in a second position or a deployed position. The dissection tip 222 is shown deployed to contact the inner portion of the wall of blood vessel 302, but is positioned prior to the plaque 304. In some embodiments, the dissection member 220 can be moved from the first position to the second position by translating the struts 226 in the longitudinal direction such that the distal ends of the struts 226 exit the distal end of the catheter body 210 and the struts 226 deflect radially away from the shaft 216. This movement and deflection can cause the dissection member 220 to expand radially with the struts until the dissection tip 222 abuts the outer wall 302 of the blood vessel 300. In some embodiments, the dissection tip 222 can expand radially such that the casing layer acts to seal the blood vessel 300 circumferentially preventing the flow of blood between the medical article 200 and the blood vessel 300. Sealing the blood vessel 300 in this way may prevent particles of atherosclerotic tissue (or other particles) from passing over the medical article 200 and subsequently embolizing within the blood vessel proximal to the dissection tip 222. Additionally, as discussed below, any particles that pass through the dissection tip 222 may be aspirated and removed from the blood vessel 300 through the medical article 200.

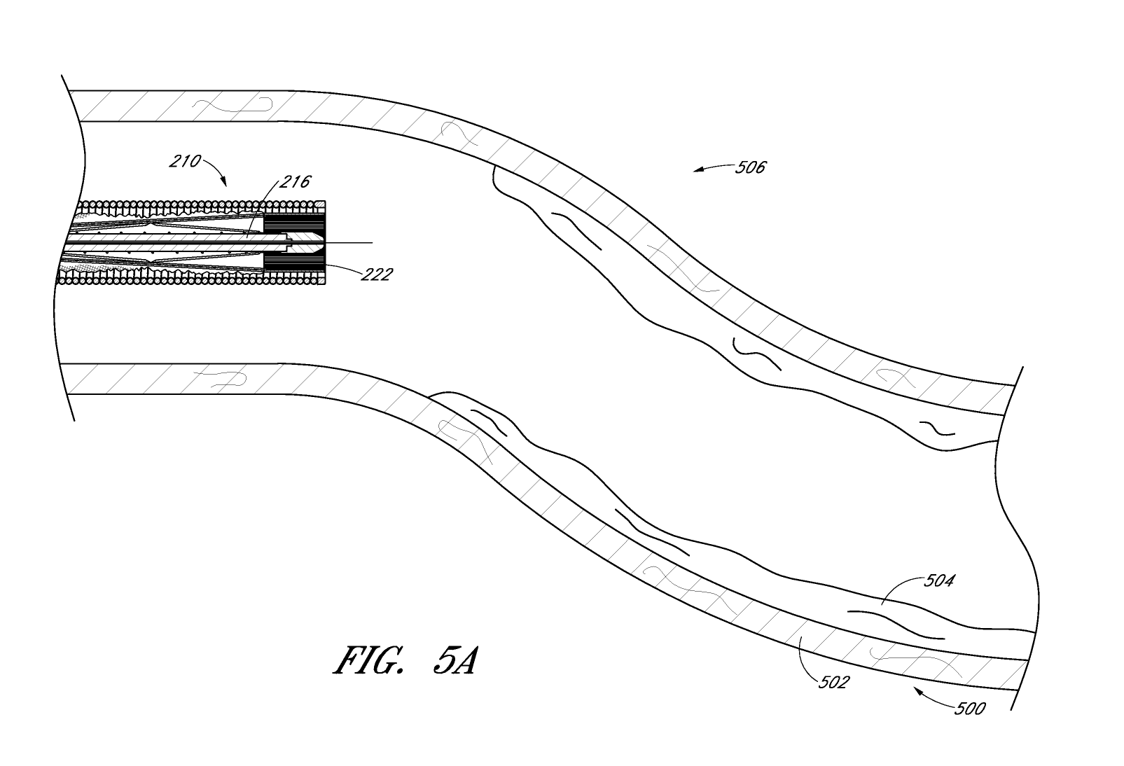

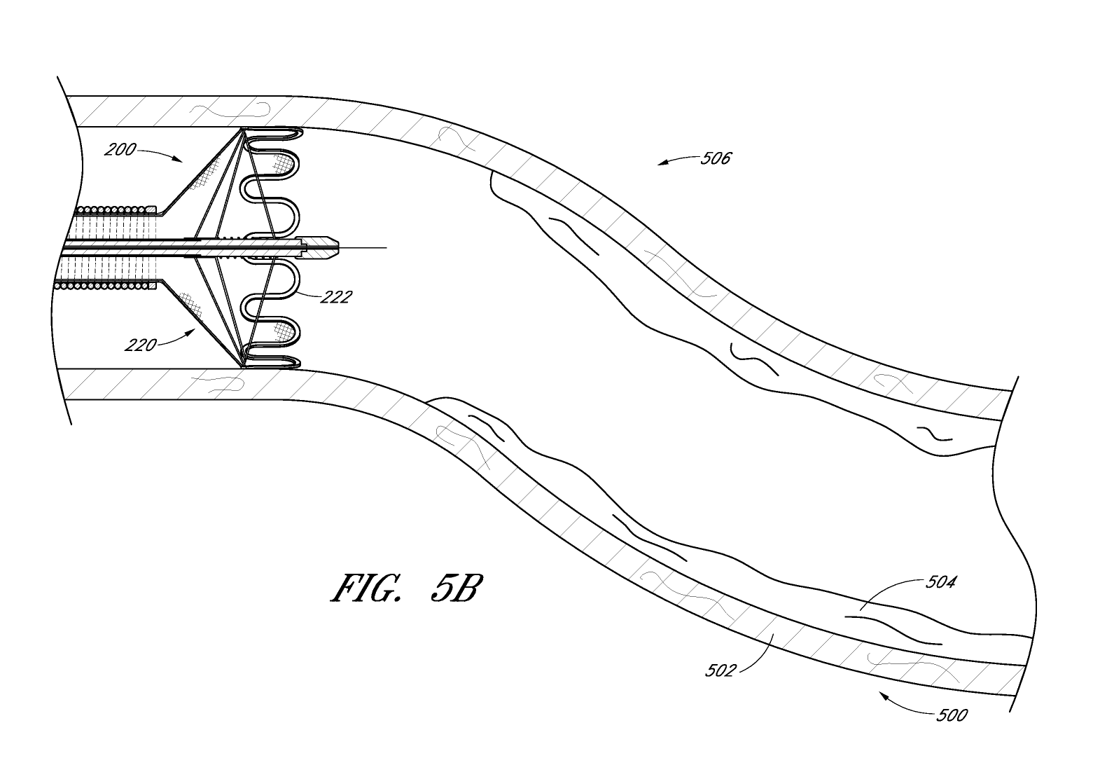

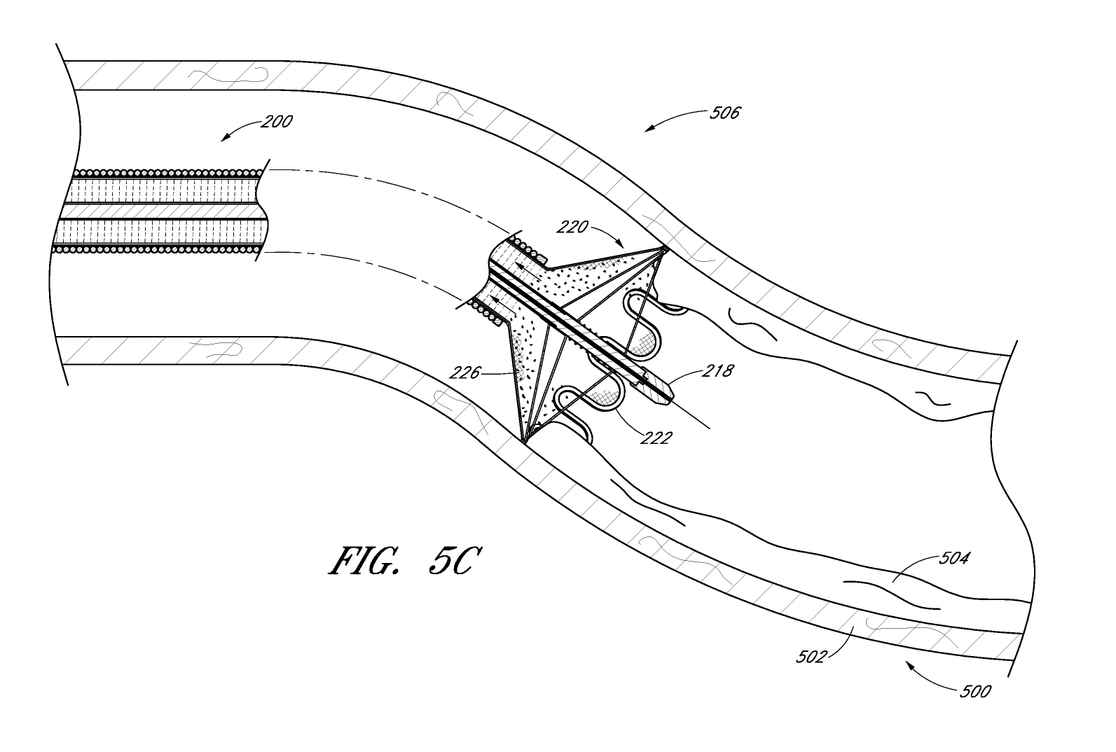

As shown in FIG. 3B, the radial deflection or movement of the distal ends of the struts 226 between FIGS. 3A and 3B can also allow the severing element 230 to expand radially such that a diameter of the severing element 230 is slightly less than a diameter of the dissection member 220. In this way, the deflection of the struts 226 can adjust the dissection tip 222 and severing element 230 to correspond to the size of the blood vessel 300. In the second position, a healthcare professional may advance the medical article 200 longitudinally such that the dissection tip 222 moves to dislodge the plaque 304, for example by traveling between the core of plaque 304 and the outer wall 302 of the blood vessel 300. It should be noted that in some aspects of this particular depiction or in any other embodiment described herein, including the embodiment below in connection with FIGS. 4-6, if desired the dissection tip 222 can be configured to move in any desired manner so as to dislodge the plaque. That movement can be longitudinally within or between any desired layer. For example, in some aspects it can be configured to move longitudinally within a subintimal layer between the plaque core and the wall of the vessel. The healthcare professional may manipulate the dissection member 220, for example, using the handle 260 described above with reference to FIG. 2E. Additionally, in some embodiments an optional ultrasonic transducer 219 disposed within the tip 218 of the medical article 200 can be used to aid a healthcare professional in positioning the dissection tip 222 between the plaque core 304 and the outer wall 302 of the blood vessel 300. Thus, the medical article 200 may be advanced with the dissection member 220 in the second position such that at least a portion or volume of the core of plaque 304 is circumferentially engulfed by the dissection member 220. It should be noted that in some aspects, the healthcare professional can utilize X-ray technology in connection with the use of the article, for example, to visualize the location of the article with respect to a stenosed portion of vasculature.

Turning now to FIG. 3C, the medical article 200 has been advanced longitudinally from its position in FIG. 3B toward the core of plaque 304. As the medical article 200 is advanced longitudinally, a volume of the core of plaque is dissected and engulfed within the dissection member 220. The dislodged core of plaque can be directed into the frusto-conical receiving space 224 defined by the casing layer 228. Upon passing through the dissection tip 222 and entering the receiving space 224, the core of plaque 304 can be severed by the severing element 230 resulting in plaque of reduced size, which as depicted are particles of plaque or atherosclerotic tissue 330. These particles 330 may be subsequently aspirated through the catheter body 210 and removed from the patient. It should be noted again that in some aspects of FIGS. 3-6 (for example) the medical article 200 can be configured with a different severing element 230, for example a non-adjustable element, or the article 200 can have no element and the dislodged material may simply be aspirated.

The medical article 200 can be advanced further in a longitudinal direction from its position in FIG. 3C to remove more of the plaque core 304 from the blood vessel 300. In some implementations, the medical article 200 can be advanced distally such that the entire plaque core 304 or substantially all of the plaque core 304 can be removed from the blood vessel 300. In other embodiments, the medical article 200 can be used to remove only a portion of a given plaque core. In embodiments where the medical article 200 is used to remove only a portion of a given plaque core, the severing element 230 may be configured to be advanced distally relative to the dissection tip 222 such that the plaque core is cut flush with the distal most edge of the dissection tip 222.

FIGS. 4A-4C are side views schematically illustrating the use of the medical article of FIGS. 2A-2E in performing an endovascular endarterectomy procedure in an example blood vessel. In contrast to FIGS. 3A-3C, the blood vessel 400 in FIGS. 4A-4C tapers or narrows in a distal direction such that an inner lumen of the blood vessel 400 bounded by an outer wall 402 of the blood vessel 400 also narrows in the distal direction. FIGS. 4A-4C illustrate, among other things, how the medical article 200 can adjust to the size of the narrowing blood vessel, thereby avoiding and/or minimizing barotraumas or other injury to the vessel. Also, illustrated is a severing element that can adjust to the narrowing size. In FIGS. 4A-4C, a plaque core 404 is disposed within a portion of the outer wall 402 of the blood vessel 400. FIG. 4A schematically illustrates the medical article 200 with the dissection tip 222 in a first position or a non-deployed position. As with FIG. 2A, in the first position the dissection tip 222 is disposed within the catheter body 210.

FIG. 4B schematically illustrates the dissection tip 222 and dissection member 220 in a second or a deployed position wherein the dissection member 220 is expanded radially from its configuration in the first position such that the dissection tip 222 abuts the outer wall 402 of the blood vessel 400. In the second position, a healthcare professional may advance the medical article 200 longitudinally such that the dissection tip 222 moves between the core of plaque 404 and the outer wall 402 of the blood vessel 400 (as discussed above, it can be positioned to travel subintimally, between the plaque core and the wall of the vessel). Thus, the medical article 200 may be advanced with the dissection member 220 in the second position such that at least a portion or volume of the core of plaque 404 is dislodged and engulfed (e.g., circumferentially dislodged and engulfed) by the dissection member 220.

Turning now to FIG. 4C, the medical article 200 is advanced longitudinally from its position in FIG. 4B toward the core of plaque 404. As the medical article 200 is advanced longitudinally, the outer wall 402 of the blood vessel narrows which can compress the dissection tip 222 radially inward. Because the dissection tip 222 and struts 226 are biased radially outward but comprise flexible materials, the dissection tip 222 can automatically adjust to the size of the inner lumen of the blood vessel 400 as the blood vessel narrows or expands longitudinally. In fact, FIG. 4C depicts a dissection tip 222 and severing element 230 that have a smaller or reduced diameter compared to the same features in FIG. 4B.

With continued reference to FIG. 4C, the medical article 200 can be advanced longitudinally in a distal direction to separate, sever, and aspirate at least a portion of the plaque core 404. In some embodiments, at least a portion of the plaque core 404 can be separated from the outer wall 402 by the dissection element, at least partially severed by the severing element 230 into particles 430, directed into the receiving space 224, and aspirated through the catheter body 210. In this way, the stenosis of the blood vessel 400 caused by the plaque core 404 can be treated endovascularly without leaving significant amounts of plaque, if any at all, in the blood vessel 400 and without applying enough pressure on the outer wall 402 that may result in barotrauma.

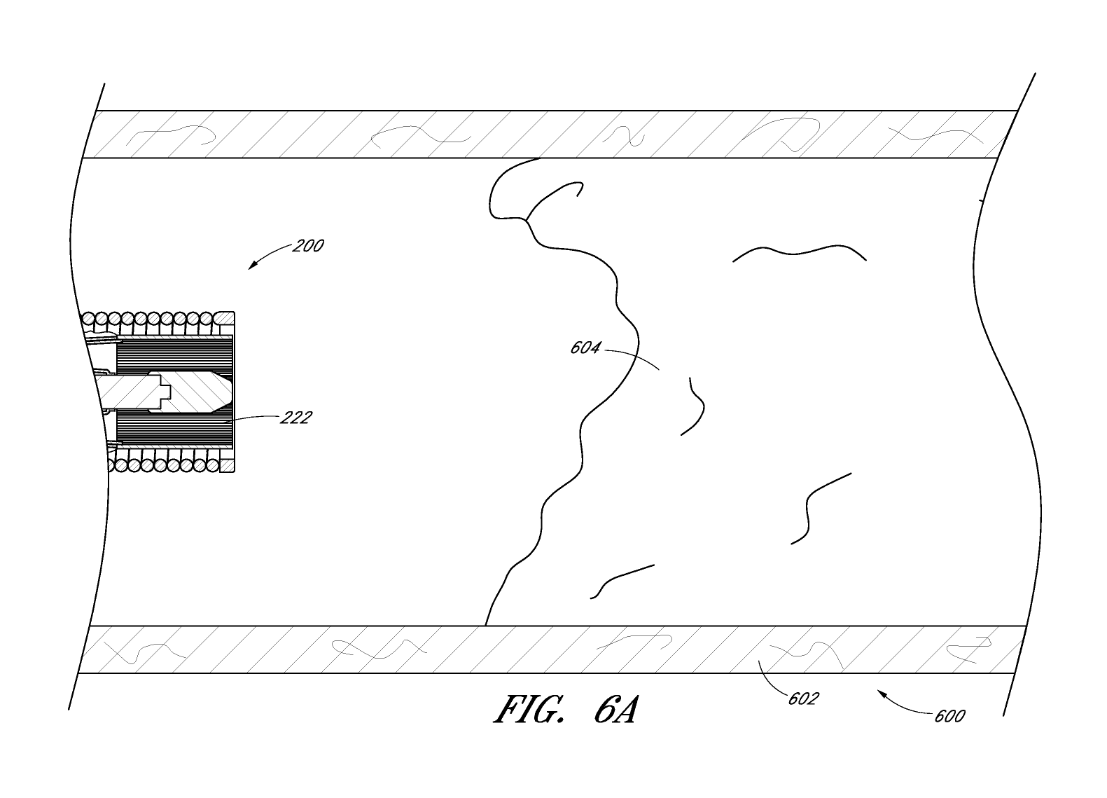

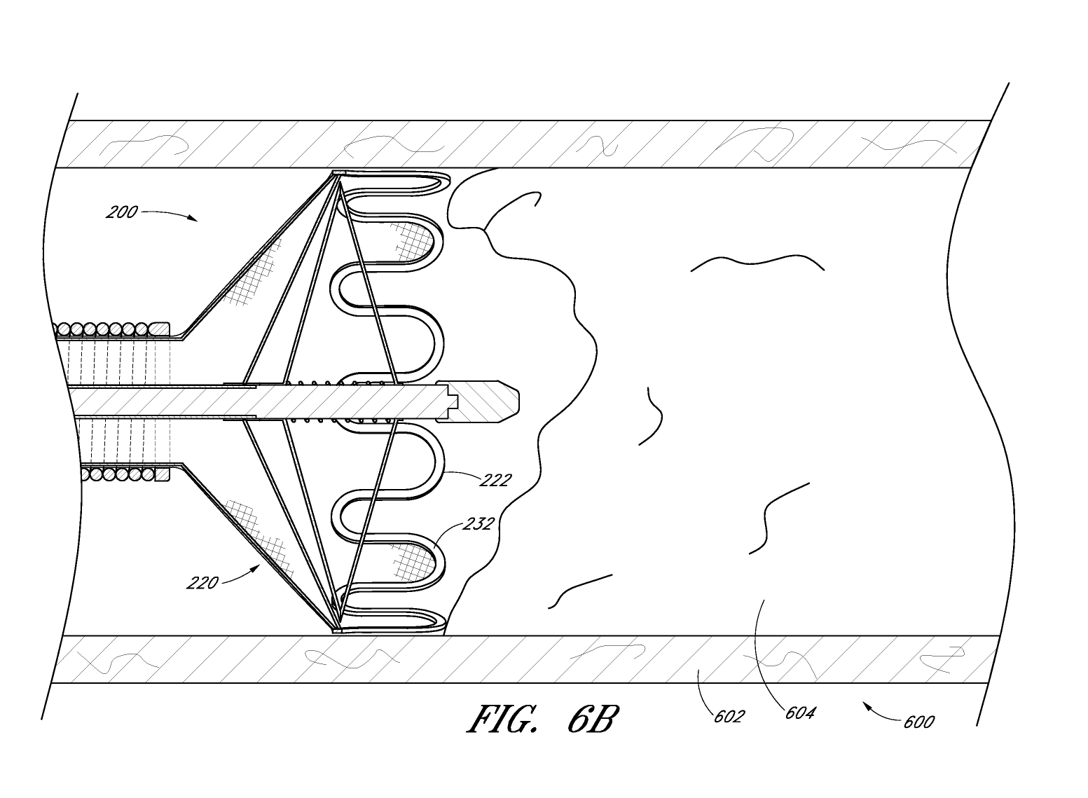

FIGS. 5A-5C are side views schematically illustrating the use of the medical article of FIGS. 2A-2E in performing an endovascular endarterectomy procedure in an example blood vessel. The example blood vessel 500 schematically illustrated in FIGS. 5A-5C includes an outer wall 502 and a plaque core 504 disposed within a turn or bent portion 506 of the blood vessel 500. FIG. 5 illustrates a medical article 200 that can maintain a proper position within a vessel that turns and that will not depart from the vessel or cause harm to the vessel. Some devices (e.g., guidewires and/or plaque removal devices) that are used in such vessels might not readily adjust to the turn or bend of the vessel, and therefore can continue on a path that will puncture or perforate the vessel wall. The configuration of the device 200 in FIG. 5 allows it to safely follow the turn or bend of the blood vessel. FIG. 5A schematically illustrates the medical article 200 with the dissection tip 222 in a first position or a non-deployed position.

FIG. 5B schematically illustrates the dissection tip 222 and dissection member 220 in a second or deployed position wherein the dissection member 220 is expanded radially from its configuration in the first position such that the dissection tip 222 abuts the outer wall 502 of the blood vessel 500. In the second position, a healthcare professional may advance the medical article 200 longitudinally such that the dissection tip 222 moves between the core of plaque 504 and the outer wall 502 of the blood vessel 500 (as discussed above, it can be positioned to travel subintimally, between the plaque core and the wall of the vessel). Thus, the medical article 200 may be advanced with the dissection tip 222 in the second position such that at least a portion or volume of the core of plaque 504 is dislodged and engulfed (e.g., circumferentially dislodged and engulfed) by the dissection member 220.

Turning now to FIG. 5C, the medical article 200 is advanced longitudinally from its position in FIG. 5B toward the core of plaque 504. As the medical article 200 is advanced longitudinally, the outer wall 502 of the blood vessel turns or bends at the turn portion 506. Because the dissection tip 222 can optionally comprise a shape memory allow that is rotatably coupled to the struts 226, the dissection tip 222 can extend substantially parallel to the longitudinal axis of the blood vessel 500 while the tip 218 of the medical article 200 is turned through the blood vessel 500. As with FIGS. 3C and 4C, the medical article 200 can be advanced longitudinally in the distal direction beyond its position in FIG. 5C to separate, sever, and aspirate at least a portion of the plaque core 504.

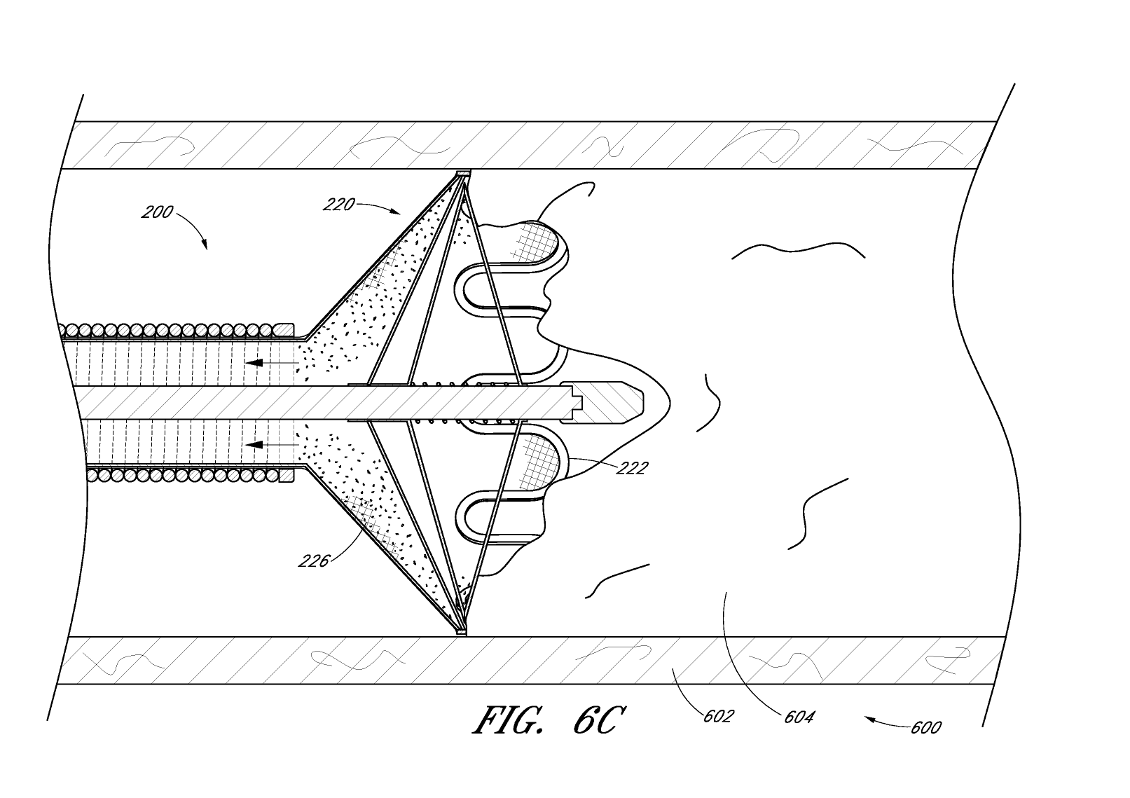

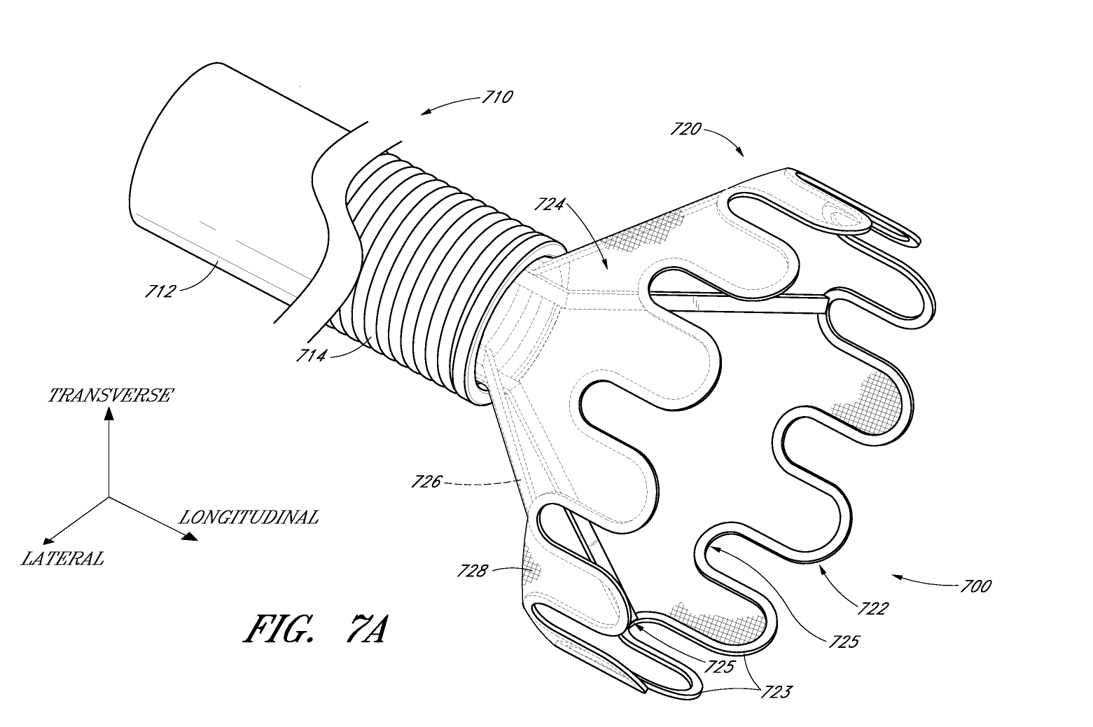

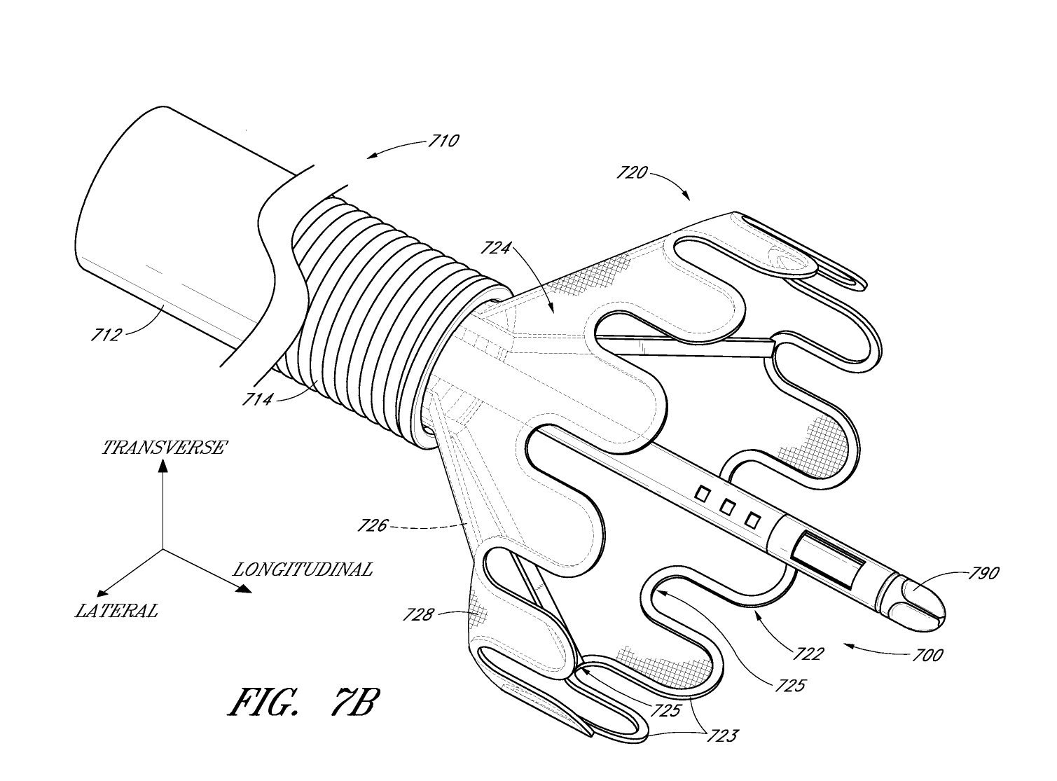

FIGS. 6A-6C depict side views schematically illustrating the use of the medical article of FIGS. 2A-2E in performing an endovascular endarterectomy procedure in an example blood vessel. The example blood vessel 600 schematically illustrated in FIGS. 6A-6C includes an outer wall 602 and a plaque core 604 that completely occludes an inner lumen of the blood vessel 600. Existing endovascular procedures for treating total occlusions of blood vessels, for example, subintimal angioplasty, can require the use of a guidewire to position a medical article in the subintimal space between a core of plaque and an outer wall of the blood vessel. However, as shown in FIGS. 6A-6C, the systems, devices, and methods disclosed herein can be implemented to remove a plaque core that is completely occluding a blood vessel without requiring the use of a guidewire. It should be noted that a guidewire can be used, but does not have to be. In some embodiments a guidewire is specifically excluded.

FIG. 6A schematically illustrates an embodiment of the medical article 200 that does not include a guidewire lumen extending therethrough. The medical article 200 in FIG. 6A is depicted as being disposed proximal to the plaque core 604 and with the dissection tip 222 in a first or non-deployed position. FIG. 6B schematically illustrates the dissection tip 222 and dissection member 220 in a second or deployed position wherein the dissection member 220 is expanded radially from its configuration in the first position such that the dissection tip 222 abuts the outer wall 602 of the blood vessel 600. In the second position, a healthcare professional may advance the medical article 200 longitudinally such that the dissection tip 222 moves between the core of plaque 604 and the outer wall 602 of the blood vessel 600 (as discussed above, it can be positioned to travel subintimally, between the plaque core and the wall of the vessel). Because the dissection tip 222 can automatically adjust to the inner diameter of the blood vessel 600 and the dissection tip 222 can comprise a flexible shape memory alloy with one or more fingers 223, the dissection tip 222 may be easily positioned in the subintimal space between the plaque core 604 and the outer wall 602. From this position, the medical article 200 may be advanced with the dissection tip 222 in the second position such that at least a portion or volume of the core of plaque 604 is dislodged and engulfed (e.g., circumferentially dislodged and engulfed) by the dissection member 220.

Turning now to FIG. 6C, the medical article 200 is advanced longitudinally from its position in FIG. 6B toward the core of plaque 604. Because the core of plaque 604 completely occludes the blood vessel 600, the distal path of the blood vessel 600 may not be readily imaged using existing vascular imaging techniques, for example, using vascular contrast agents. As such, it may be impossible to guide an intravascular medical device, for example, a guidewire or the medical article 200 of FIGS. 2A-2E, distally within the blood vessel 600 using extracorporeal images because the path of the blood vessel may not be apparent. Accordingly, existing endovascular methods of treating complete occlusions may result in perforations of the outer wall of the blood vessel by a medical article. However, as the medical article 200 is advanced distally, the dissection tip 222 and struts 226 can automatically adjust to the inner surface of the outer wall 602 of the blood vessel 600. Additionally, as discussed above with reference to FIGS. 5A-5C, the dissection tip 222 can constantly extend parallel to the longitudinal axis of the blood vessel 600. Therefore, the medical article 200 can be used to separate, sever, and aspirate a plaque core that completely occludes blood vessel 600 without the use of a guidewire and without imaging capabilities to navigate the medical article 200 within the blood vessel 600.

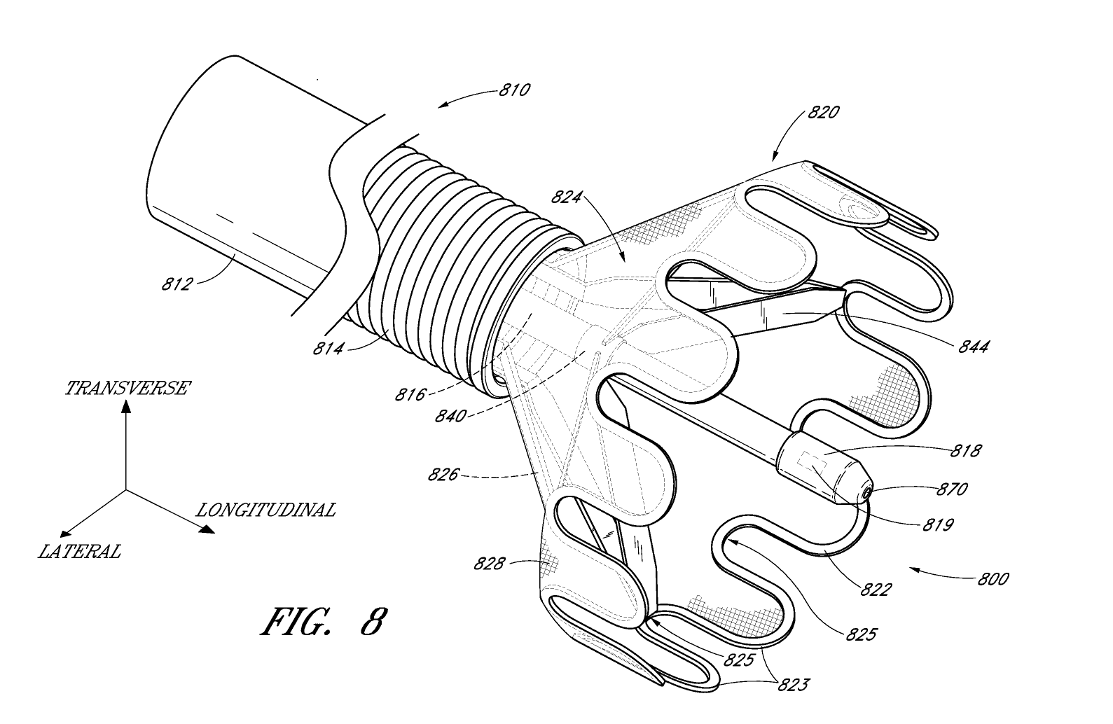

FIG. 7A depicts a perspective view of a portion of one non-limiting example of an embodiment of a medical article 700 for use in treating a patient. The medical article 700 of FIG. 7A includes a catheter body 710 and a dissection member 720 configured to move radially and longitudinally relative to the catheter body 710. The catheter body 710 can comprise an elongated tubular shape defining one or more internal lumens. In some embodiments, the catheter body 710 can be flexible enough to be steered through a tortuous portion of a patient's vasculature yet may be rigid enough to be pushed distally through a given lumen. Thus, in some embodiments, the catheter body 710 can include a flexible coil body 714. The catheter body can optionally by coated, for example, by a hydrophilic coating 712.

Similar to the dissection member 220 discussed above with reference to FIGS. 2-6, the dissection member 720 can be configured to adjust radially (e.g., to expand or contract radially) from the longitudinal axis of the medical article 700 between at least a first position and a second position (shown in FIG. 7A). In some embodiments, the dissection member 720 can be at least partially disposed within the catheter body 710 when it is in the first position and can be disposed distal to a distal end of the catheter body 710 when it is in the second position. The dissection member 720 can include a dissection tip 722 and a receiving space 724 disposed proximal thereto. The dissection tip 722 can define an opening that provides ingress and egress to the receiving space 724. In some embodiments, the opening can be circular, oval, or irregularly shaped and can be defined by, and/or conform to, the shape of a blood vessel that the dissection tip 722 is disposed within.

In some embodiments, the dissection tip 722 can optionally have one or more fingers 723 that extend substantially parallel to one another and substantially parallel to the longitudinal axis of the medical article 700. In use, the fingers 723 can extend substantially parallel to the walls of a blood vessel such that the medical article 700 can be moved longitudinally relative to the blood vessel. In some embodiments, the fingers 723 can be curvilinearly shaped and can be connected to one another by one or more arc segments 725. However, in other embodiments, the fingers 723 can be differently shaped, for example, they may be polygonal and connected to one another by curved segments and/or linear segments. In some embodiments, the dissection tip 722 may not comprise fingers and may be differently shaped, for example, the dissection tip 722 can include teeth, wedges, or other objects that are shaped differently than the fingers 723. Additionally, in other embodiments, the distal end of the dissection tip 722 can be planar. In some embodiments, the dissection tip 722 can comprise a flexible alloy, for example, a nickel-titanium alloy, such that the dissection tip 722 is somewhat rigid yet can expand and flex radially between the first position and the second position.

Still referring to FIG. 7A, in some embodiments, the medical article 700 can include one or more struts 726. The struts 726 can be configured to move longitudinally relative to the catheter body 710 and can be disposed at least partially within the catheter body 710 as shown. For example, in one embodiment, the struts 726 can slide within the catheter body 710. In some embodiments, the struts 726 can comprise a flexible metal, for example, steel, stainless steel, or spring steel, having a pre-formed memory such that the distal ends of the struts 726 are configured to deflect radially away from the longitudinal axis of the medical article 700 when the struts 726 are extended distally from the distal end of the catheter body 710 (e.g., when the distal ends of the struts 726 are not bounded by the catheter body 710).

The struts 726 can be optionally coupled to a proximal end of the dissection member 720. In some embodiments, a hinge, for example a living hinge, can be disposed between the struts 726 and the dissection tip 722 to rotatably or hingedly couple the struts 726 to the dissection member 720. In this way, distal ends of the struts 726 may be completely disposed within the catheter body 710 when the dissection member 720 is in the first position and the distal ends of the struts 726 can be translated longitudinally through the distal end of the catheter body 710 such that the distal ends of the struts 726 deflect radially away from the longitudinal axis of the medical article 700 upon passing through the distal end of the catheter body 710. The longitudinal movement and radial deflection of the struts 726 relative to the longitudinal axis of the medical article 700 can move the dissection tip 722 between at least the first position and the second position (illustrated in FIG. 7A). Thus, when the struts 726 are moved distally relative to the catheter body 710 within a blood vessel, the struts 726 and the dissection tip 722 may expand radially to contact the inner wall of the blood vessel. In some embodiments, the struts can have a pre-formed memory such that the dissection tip 722 automatically expands to the boundaries of the inner lumen of a blood vessel (e.g., to the surface of the inner wall of the blood vessel) and the size of the dissection tip 722 can vary with the blood vessel as the medical article 700 is advanced and/or retracted therethrough.

With continued reference to FIG. 7A, an optional casing layer 728 can be disposed circumferentially around the struts 726 and at least a portion of the dissection member 720. The casing layer 728 can comprise various flexible materials, for example, materials such as latex and/or Mylar, and can comprise various non-flexible materials, for example, rigid composites. In some embodiments, the casing layer 728 can extend along the longitudinal length of the struts 726 and in other embodiments, the casing layer 728 can extend along a portion of the longitudinal length of the struts 726 that is less than an entire longitudinal length of the struts 726. When the dissection member 720 is in the second position (shown in FIG. 7A), the casing layer 728 can at least partially define the receiving space 724 between the distal end of the dissection tip 722 and the distal end of the catheter body 710. The receiving space 724 can be defined by various shapes including, for example, frusto-conical shapes, conical shapes, and/or frustums.

In use the medical article 700 can be used to separate a core of plaque from a patient's vasculature. For example, in one embodiment, the medical article 700 may be advanced and/or retracted through a patient's vasculature with the dissection member 720 in the first (or non-deployed) position. The dissection member 720 may then be manipulated to the second (or deployed) position such that the dissection tip 722 radially adjusts to the size of the blood vessel. When the dissection member 720 is expanded radially to the second position, the dissection tip 722 can be maneuvered between a core of plaque and the outer wall of the blood vessel that the medical article 700 is disposed within. From this configuration, the medical article 700 can be advanced distally through the patient's vasculature such that the dissection tip 722 dissects or separates at least a portion of the core of plaque from the outer wall. The dissected or separated plaque may be engulfed within the dissection member 720 and can pass through the dissection tip 722 to the receiving space 724.

The medical article 700 can be used as a stand-alone device to separate or dissect plaque from a patient's vasculature and/or can be used in conjunction with other devices and/or components. In some embodiments, the medical article 700 can further comprise additional components, for example, one or more aspiration lumens, one or more severing elements, one or more ultrasound elements, and/or a guidewire. Similarly, in some embodiments, the medical article 700 can be configured to receive or interact with other medical devices. For example, the medical article 700 can be configured to receive at least a portion of an endovascular atherectomy device such that plaque that has been separated and engulfed within the dissection member 720 can be further processed by the one or more other medical devices. For example, in some embodiments, the medical article 700 can be used in conjunction with one or more atherectomy devices available from Pathway Medical Technologies of Kirkland, Wash. FIG. 7B depicts an example of such a device 790 used in conjunction with the medical article 700. Although the atherectomy device 790 in FIG. 7B is schematically depicted as extending beyond the dissection member 720 of the medical article 700, it will be appreciated by those of skill in the art that the atherectomy device 790 may also by positioned differently in relation to the medical article. For example, the atherectomy device 790 may be positioned such that the cutting tip of the atherectomy device 790 is even with the distal most end of the dissection member 720 or the atherectomy device 790 may be positioned such that the cutting tip of the atherectomy device 790 is proximal to the distal most end of the dissection member 720. Also, in some embodiments, the medical article 700 can be used with other devices that are used for removal of clots or other vessel obstructions. In embodiments where the article 700 is used with other devices, the medical article 700 can be provided to a healthcare provider alone and/or in a kit including one or more of the other medical articles that may be used in a given procedure. Of course, it will also be appreciated by those of skill in the art that the medical article 700 is just one example of a structure that may be configured to adjust to the size of a blood vessel in order to facilitate the dislodging, dissection, and/or separation of material (e.g., plaque, calcified material, intima tissue, etc.) from the blood vessel.

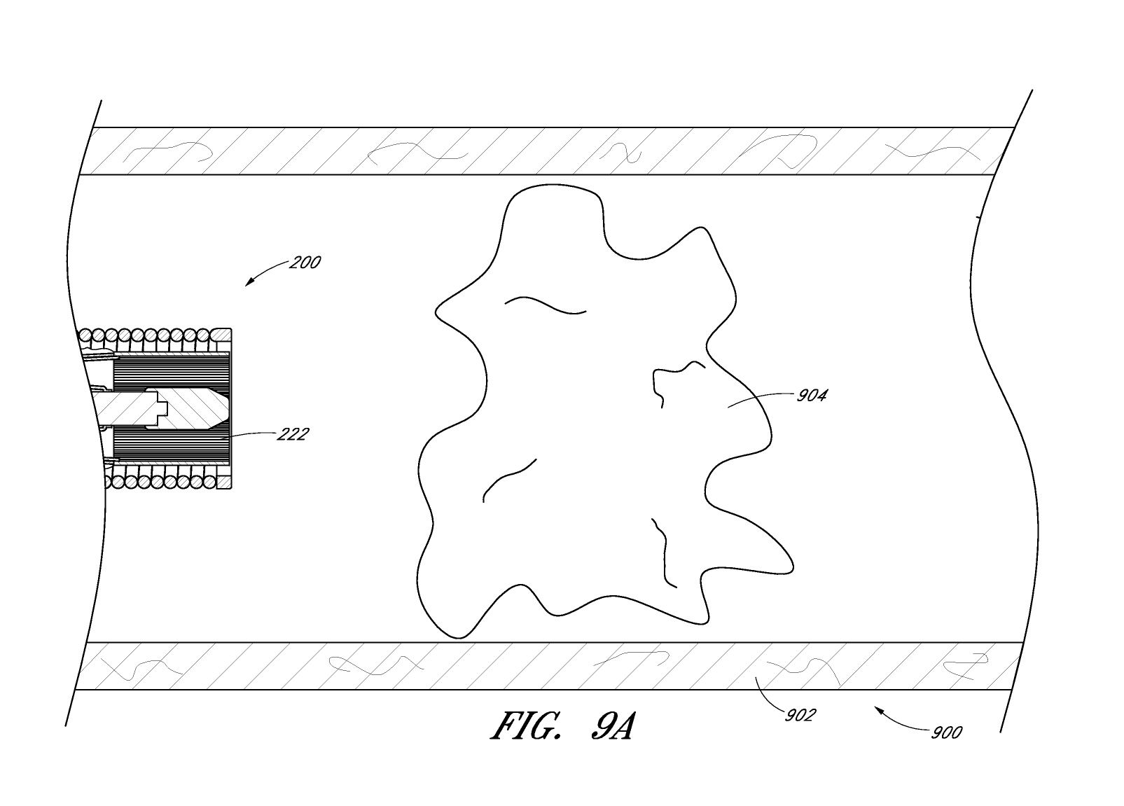

FIG. 8 depicts a perspective view of a portion of another non-limiting example of an embodiment of a medical article 800 for use in treating a patient. The medical article 800 of FIG. 8 includes a catheter body 810 and a dissection member 820 configured to move relative to the catheter body 810. The catheter body 810 can comprise an elongated tubular shape defining one or more internal lumens. In some embodiments, the catheter body 810 can be flexible enough to be steered through a tortuous portion of a patient's vasculature yet may be rigid enough to be pushed distally through a given lumen. Thus, in some embodiments, the catheter body 810 can include a flexible coil body 814. The catheter body can optionally by coated, for example, by a hydrophilic coating 812.

Similar to the dissection member 220 discussed above with reference to FIGS. 2-6, the dissection member 820 can be configured to adjust radially (e.g., to expand or contract radially) from the longitudinal axis of the medical article 800 between at least a first position and a second position (shown in FIG. 8). Further, the dissection member 820 can be configured to rotate about the longitudinal axis of the medical article 800 relative to the catheter body 810. In some embodiments, the dissection member 820 can be at least partially disposed within the catheter body 810 when it is in the first position and can be disposed distal to a distal end of the catheter body 810 when it is in the second position. The dissection member 820 can include a dissection tip 822 and a receiving space 824 disposed proximal thereto.

In some embodiments, the dissection tip 822 can optionally have one or more fingers 823 that extend substantially parallel to one another and substantially parallel to the longitudinal axis of the medical article 800. In use, the fingers 823 can extend substantially parallel to the walls of a blood vessel such that the medical article 800 can be moved longitudinally relative to the blood vessel. In some embodiments, the fingers 823 can be curvilinearly shaped and can be connected to one another by one or more arc segments 825.

Still referring to FIG. 8, in some embodiments, the medical article 800 can include one or more struts 826. The struts 826 can be configured to move longitudinally relative to the catheter body 810 and can be disposed at least partially within the catheter body 810 as shown. For example, in one embodiment, the struts 826 can slide within the catheter body 810. In some embodiments, the struts 826 can comprise a flexible metal, for example, steel, stainless steel, or spring steel, having a pre-formed memory such that the distal ends of the struts 826 are configured to deflect radially away from the longitudinal axis of the medical article 800 when the struts 826 are extended distally from the distal end of the catheter body 810 (e.g., when the distal ends of the struts 826 are not bounded by the catheter body 810).

The struts 826 can be optionally coupled to a proximal end of the dissection member 820. In some embodiments, a hinge, for example a living hinge, can be disposed between the struts 826 and the dissection tip 822 to rotatably or hingedly couple the struts 826 to the dissection member 820. In this way, distal ends of the struts 826 may be completely disposed within the catheter body 810 when the dissection member 820 is in the first position and the distal ends of the struts 826 can be translated longitudinally through the distal end of the catheter body 810 such that the distal ends of the struts 826 deflect radially away from the longitudinal axis of the medical article 800 upon passing through the distal end of the catheter body 810. The longitudinal movement and radial deflection of the struts 826 relative to the longitudinal axis of the medical article 800 can move the dissection tip 822 between at least the first position and the second position (illustrated in FIG. 8). Thus, when the struts 826 are moved distally relative to the catheter body 810 within a blood vessel, the struts 826 and the dissection tip 822 may expand radially to contact the inner wall of the blood vessel.

Also schematically depicted in FIG. 8 are connection members 844 that couple each of the struts 826 to a slidable collar 840. Each of the connection members 844 can comprise a rigid material, for example, steel, stainless steel, or spring steel, can be rotatably coupled at one end to a strut 826, and can be rotatably coupled at an opposite end to the slidable collar 840. Thus, the connection members 844 can serve to indirectly couple the slidable collar 840 with the struts 826. In this way, radial expansion or outward deflection of the struts 826 relative to the longitudinal axis of the medical article 800 can slide the slidable collar 840 distally along a shaft 816.

In contrast to the medical article 200 discussed above with reference to FIGS. 1-6, the medical article 800 does not include a severing element that rotates relative to the dissection tip 822. Instead, the dissection tip 822 can be rotated relative to the catheter body 810 by rotatably driving the shaft 816. Because the shaft 816 can be secured relative to the dissection tip 822 via the slidable collar 840 and connection elements 844, rotation of the shaft within the catheter body 810 can result in the rotation of the dissection tip 822. Additionally, the dissection tip 822 can be optionally rotated by rotatably driving one or more of the struts 826. In this embodiment, the connection members 844 can include one or more edges or blades capable of severing tissue within a patient's vasculature (e.g., plaque) once the tissue is received within the receiving space 824.

With continued reference to FIG. 8, an optional casing layer 828 can be disposed circumferentially around the struts 826 and at least a portion of the dissection member 820. The casing layer 828 can comprise various flexible materials, for example, materials such as latex and/or Mylar, and can comprise various non-flexible materials, for example, rigid composites. In some embodiments, the casing layer 828 can extend along the longitudinal length of the struts 826 and in other embodiments, the casing layer 828 can extend along a portion of the longitudinal length of the struts 826 that is less than an entire longitudinal length of the struts 826. When the dissection member 820 is in the second position (shown in FIG. 8), the casing layer 828 can at least partially define the receiving space 824 between the distal end of the dissection tip 822 and the distal end of the catheter body 810. The receiving space 824 can be defined by various shapes including, for example, frusto-conical shapes, conical shapes, and/or frustums.

Still referring to FIG. 8, in some embodiments the medical article 800 also optionally can include a tip 818. The tip 818 may include, for example, one or more ultrasonic transducers 819. The tip 818 may include, for example, a distal guidewire aperture 870 that provides access to a guidewire lumen. As depicted, the tip 818 can include both the one or more transducers 819 and the guidewire aperture 870, but in some embodiments may include neither of those elements, one of those elements, both elements and/or additional elements. The optional distal guidewire aperture 870 can provide ingress and egress to an optional guidewire lumen in the shaft 816 to allow an optional guidewire to slide in and out of the shaft 816. The tip 818 can be disposed at a distal end of the shaft 816 and can extend distal to the distal most edge of the dissection tip 822 or can be disposed proximal to the distal most edge of the dissection tip 822.

In some embodiments, the one or more ultrasonic transducers 819 can be part of an intravascular ultrasound system configured to image portions of a blood vessel that the medical article 800 may be inserted into. In such embodiments, the intravascular ultrasound system can be side looking (e.g., radial to the longitudinal axis of the medical article 800) and/or forward looking (e.g., parallel to the longitudinal axis of the medical article 800). In some embodiments, the shaft 816 can include one or more conductive elements, for example, one or more wires, such that signals may be sent and received by the one or more transducers 89 to control circuitry located proximal to the tip 818 (e.g., proximal to the medical article 800). Thus, the one or more ultrasonic transducers 819 and an associated intravascular ultrasound system can enable a health care professional to position the medical article 800 and dissection member 820 relative to a patient's blood vessel. For example, the one or more ultrasonic transducers 819 can be utilized to position the dissection tip 822 circumferentially around a core of plaque and between the plaque and a wall of a blood vessel.

In use the medical article 800 can be used to separate a core of plaque from a patient's vasculature. For example, in one embodiment, the medical article 800 may be advanced and/or retracted through a patient's vasculature with the dissection member 820 in the first (or non-deployed) position. The dissection member 820 may then be manipulated to the second (or deployed) position such that the dissection tip 822 radially adjusts to the size of the blood vessel. When the dissection member 820 is expanded radially to the second position, the dissection tip 822 can be maneuvered between a core of plaque and the outer wall of the blood vessel that the medical article 800 is disposed within. From this configuration, the medical article 800 can be advanced distally through the patient's vasculature such that the dissection tip 822 dissects or separates at least a portion of the core of plaque from the outer wall. The dissected or separated plaque may be engulfed within the dissection member 820 and can pass through the dissection tip 822 to the receiving space 824. Once within the receiving space 824, the connection elements 844 can be rotated relative to the catheter body 810 (along with the dissection tip 822) to sever the engulfed plaque from the patient.

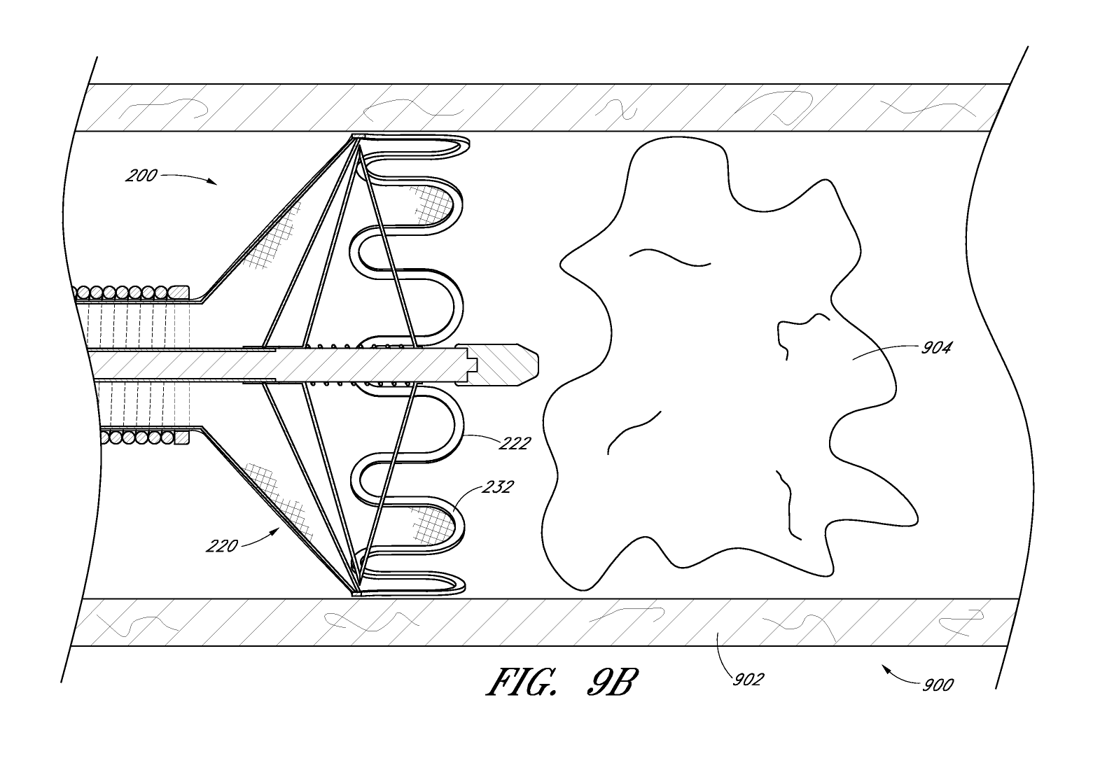

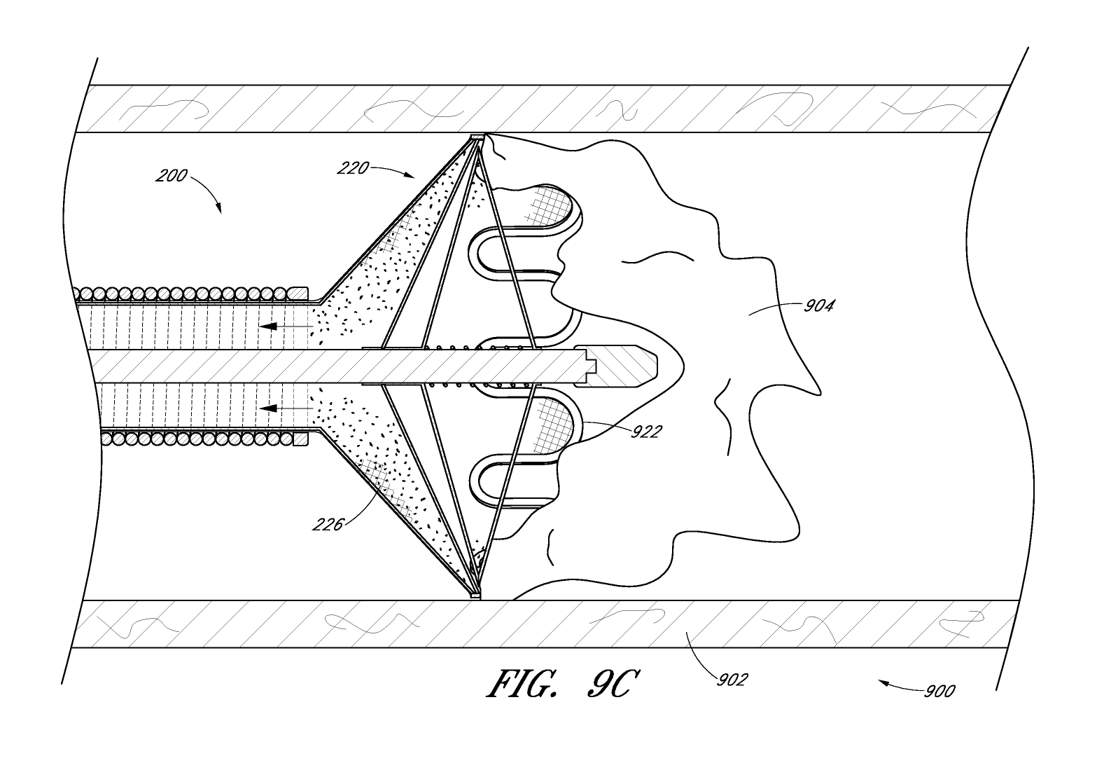

As discussed above with reference to FIGS. 1-8, embodiments of medical articles disclosed herein can be used to remove plaque from a patient's blood vessel to treat one or more stenosed sections of the patient's vasculature. As shown in FIGS. 9A-9C, the embodiments of medical articles disclosed herein can also be used to remove other objects from a patient's vasculature, for example, a thrombus or blood clot, including for example, those that result from or occur in connection with the implantation and use of a fistula.

One existing method for blood cot removal includes open surgery where a healthcare professional makes an incision to open a clotted vessel and subsequently guides a deflated balloon through the vessel past the thrombus or clot. The healthcare professional may then inflate the balloon and pull the inflated balloon towards the incision to remove the clot through the open incision in the vessel. However, such a method requires surgery which may result in higher risks, bleeding, scars, and/or pain etc. Another method for treating a clot is pharmacologic thrombolysis which includes using an infusion catheter to introduce clot dispersing drugs into a vessel to disperse a given clot. However, this method can leave pieces of clot behind in the patient's vasculature and these remnant pieces can become lodged in smaller vessels of the patient's vasculature resulting in necrosis. Additional methods for treating clots, for example, mechanical thrombectomy procedures or ultrasound treatment procedures, may also leave portions of a clot behind in the patient's vasculature. As discussed in further detail below with reference to FIGS. 9A-9C, embodiments of medical articles disclosed herein can be utilized to completely remove a clot or thrombus from a patient's vasculature and may be introduced endovascularly through a small incision without requiring open surgery.

The example blood vessel 900 schematically illustrated in FIGS. 9A-9C includes an outer wall 802 and a clot or thrombus 804 disposed within an inner lumen of the blood vessel 900. In some instances, a clot or thrombus may occlude or partially occlude a portion of a blood vessel and restrict the flow of blood through the clotted section. FIG. 9A schematically illustrates an embodiment of the medical article 200 discussed above with reference to FIGS. 1-6. The medical article 200 in FIG. 9A is depicted as being disposed proximal to the clot 904 and with the dissection tip 222 in a first or non-deployed position. FIG. 9B schematically illustrates the dissection tip 222 and dissection member 220 in a second or deployed position wherein the dissection member 220 is expanded radially from its configuration in the first position such that the dissection tip 222 abuts the outer wall 902 of the blood vessel 900. In the second position, a healthcare professional may advance the medical article 200 longitudinally such that the dissection tip 222 moves between the clot 904 and the outer wall 902 of the blood vessel 900. Because the dissection tip 222 can automatically adjust to the inner diameter of the blood vessel 900 and the dissection tip 222 can comprise a flexible shape memory alloy with one or more fingers 223, the dissection tip 222 may be easily positioned between the plaque core 904 and the outer wall 902. From this position, the medical article 200 may be advanced with the dissection tip 222 in the second position such that the clot 904 is dislodged from the vessel 900 and engulfed (e.g., circumferentially dislodged and engulfed) by the dissection member 220.

Turning now to FIG. 9C, the medical article 200 is advanced longitudinally from its position in FIG. 9B toward the clot 904. As the medical article 200 is advanced longitudinally, the clot 904 is removed from the vessel 900 and engulfed within the dissection member 220. The removed clot 904 can be directed into the frusto-conical receiving space 224 defined by the casing layer 228. Upon passing through the dissection tip 222 and entering the receiving space 224, the clot 904 can optionally be severed by the severing element 230 resulting in plaque of reduced size, which as depicted are particles of plaque or atherosclerotic tissue 930. These particles 930 may be subsequently aspirated through the catheter body 210 and removed from the patient. It should be noted again that in some aspects of FIGS. 9A-9C (for example) the medical article 200 can be configured with a different severing element 230, for example a non-adjustable element, or the article 200 can have no element and the dislodged material may simply be aspirated.

The foregoing description details certain embodiments of the systems, devices, and methods disclosed herein. It will be appreciated, however, that no matter how detailed the foregoing appears in text, the devices and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated. The scope of the disclosure should therefore be construed in accordance with the appended claims and any equivalents thereof.

It will be appreciated by those skilled in the art that various modifications and changes may be made without departing from the scope of the described technology. Such modifications and changes are intended to fall within the scope of the embodiments, as defined by the appended claims. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures may be combined, interchanged or excluded from other embodiments.

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method comprising:
introducing a medical article into a patient's vasculature, the medical article comprising a radially adjustable dissection tip, a receiving space disposed proximal to the dissection tip, and an aspiration lumen disposed proximal to the receiving space;
positioning the dissection tip between a volume of plaque tissue or a blood clot and an outer wall of the patient's vasculature; and
receiving the volume of plaque tissue or blood clot in the receiving space.

2. The method of claim 1, further comprising advancing the medical article distally through the patient's vasculature.

3. The method of claim 2, wherein the medical article is advanced distally over a guidewire.

4. The method of claim 2, wherein the medical article is advanced distally without the use of a guidewire.

5. The method of claim 1, wherein the medical article further comprises a severing element.

6. The method of claim 5, wherein the severing element has a longitudinal length that does not extend distally beyond the most distal edge of the dissection tip.

7. The method of claim 5, wherein the severing element is configured to expand radially when the dissection tip is expanded radially.

8. The method of claim 7, wherein the severing element is configured to rotate relative to the dissection tip when the dissection tip is expanded radially.

9. The method of claim 5, wherein the severing element does not expand.

10. The method of claim 1, further comprising severing the received volume of plaque tissue or blood clot from the patient's vasculature.

11. The method of claim 10, further comprising aspirating at least a portion of the received volume of plaque tissue through an aspiration lumen.

12. The method of claim 1, wherein positioning the dissection tip between the volume of plaque tissue or the blood clot and the outer wall of the patient's vasculature includes positioning the dissection tip such that the dissection tip is disposed circumferentially about the volume of plaque tissue or the blood clot.

13. A medical article comprising:
a catheter body having a distal end, a proximal end, and a longitudinal axis extending therebetween; and
a dissection tip configured to move between at least a first position and a second position, wherein the dissection tip is disposed at least partially within the catheter body in the first position, wherein the dissection tip is disposed distal to the distal end of the catheter body in the second position, and wherein the dissection tip is biased to expand radially from the longitudinal axis when the dissection tip is moved from the first position to the second position.

14. The medical article of claim 13, further comprising a severing element configured to expand radially from the longitudinal axis when the dissection tip is moved from the first position to the second position.

15. The medical article of claim 14, wherein the severing element is configured to rotate relative to the catheter body when the dissection tip is in the second position.

16. The medical article of claim 15, wherein the severing element is configured to rotate relative to the dissection tip when the dissection tip is in the second position

17. The medical article of claim 14, wherein at least a portion of the severing element is disposed within the catheter body when the dissection tip is in the first position.

18. The medical article of claim 14, wherein the severing element is disposed proximal to a most distal point of the dissection tip when the dissection tip is in the second position.

19. The medical article of claim 18, wherein at least a portion of the severing element is disposed distal to the catheter body when the dissection tip is in the second position.

20. The medical article of claim 13, further comprising an ultrasonic transducer configured to transmit ultrasound energy.

21. The medical article of claim 20, wherein at least a portion of the ultrasonic transducer is disposed distal to the catheter body when the dissection tip is in the second position.

22. The medical article of claim 13, wherein the dissection tip is configured to adjust its radial size according to the size of a blood vessel into which it is introduced.

23. The medical article of claim 22, wherein the dissection tip is configured to decrease its radial size as it is moved into a blood vessel that is more narrow in radial size.

24. The medical article of claim 14, further comprising a plurality of struts coupled to the dissection tip.

25. The medical article of claim 13, wherein the dissection tip extends substantially parallel to the longitudinal axis when the dissection tip is in the second position.

26. The medical article of claim 13, further comprising a shaft extending at least partially through the catheter body and parallel to the longitudinal axis.

27. The medical article of claim 26, wherein at least a portion of the severing element is configured to slide along at least a portion of the shaft.

28. The medical article of claim 27, wherein the severing element comprises a first collar configured to slide along at least a portion of the shaft.

29. The medical article of claim 28, wherein the first collar is configured to rotate about the shaft.

30. The medical article of claim 26, wherein the shaft comprises a guidewire lumen extending at least partially therethrough.

31. The medical article of claim 26, wherein the shaft comprises a drive shaft configured to rotate the severing element relative to the catheter body.
